(19) United States
(12) Patent Application Publication
(10) Pub. No.: US 2024/0307591 A1
(43) Pub. Date: Sep. 19, 2024

(54) Accelerated Development of Functional Three-Dimensional Tissue Moduli

(71) Applicant: National University of Ireland, Galway, Galway (IE)

(72) Inventors: Dimitrios Zeugolis, GALWAY (IE); Kyriakos Spanoudes, GALWAY (IE); Stefanie Korntner, GALWAY (IE); Salome Guillaumin, GALWAY (IE); Elena De Lucia, GALWAY (IE); Yuri Rochev, GALWAY (IE); Andrea De Pieri, GALWAY (IE); Shubhasmin Rana, GALWAY (IE); Mehmet Gurdal, GALWAY (IE)

(21) Appl. No.: 18/577,960

(22) PCT Filed: Jul. 11, 2022
(86) PCT No.: PCT/EP2022/069359

§ 371 Date: Jan. 9, 2024

(30) Foreign Application Priority Data
Jul. 9, 2021 (EP) 21184911.2

(51) Int. Cl. A61L 27/38; A61L 27/26

(52) U.S. Cl. CPC A61L 27/3834; A61L 27/26; A61L 27/3895

(57) ABSTRACT

Accelerated development of functional three-dimensional tissue moduli. The present invention relates to a process for the production of two and three-dimensional tissues and to tissues produced by the method. The present invention further relates to a process for tissue production using polyacrylic acid as a macromolecular crowder and to tissues produced by the method.

### FIELD OF THE INVENTION

The present invention relates to a process for the production of two and three-dimensional tissues and to tissues produced by the method. The present invention further relates to a process for tissue production using polyacrylic acid as a macromolecular crowder and to tissues produced by the method.

### BACKGROUND TO THE INVENTION

Cell-based therapies, either as cell-alone injections or as cell-scaffold combinations, are at the forefront of scientific research and technological innovation in regenerative medicine to treat, augment or replace a lost, due to disease or injury, tissue function. Despite the numerous positive preclinical and clinical results in a diverse range of clinical indications, the mode of administration of injected cell suspensions offers poor control over cell protection and localisation at the site of implantation and the use of artificial scaffolds triggers adverse immune responses. To overcome these limitations, the concept of scaffold-free in vitro organogenesis has been pioneered that allows for the production of tissue-like assemblies in vitro, by exploiting the inherent ability of cells to synthesise and deposit their own extracellular matrix (ECM).

Traditionally, the production of three-dimensional (3D) scaffold-free tissue equivalents is a two-step process. Firstly, single cell sheets are produced using surfaces coated with temperature-responsive polymers, which upon reduction of the temperature below the lower critical solution temperature (LCST) of the polymer allow for the detachment of intact cells and deposited ECM thin layers. Then, by layering multiple cell sheets together and culturing them for the required maturation time, 3D tissue-like assemblies are produced. Despite the positive outcomes, with respect to safety and therapeutic efficacy, of scaffold-free in vitro organogenesis approaches in preclinical and clinical setting, only a handful of products have been commercialised. This limited technology transfer from laboratories to clinics has been attributed to numerous factors. For example, it is still technically challenging to produce multi-layered cell sheets in reproducible fashion. The creation of thick enough multi-layered cell sheets not only requires millions of cells, frequently not available for autologous applications, but also is associated with limited nutrient, waste and oxygen transfer in the middle layers, which results in cell death and delamination. Multiple operations of up to 3 layers are recommended, based on preclinical data, but again this approach requires high cell numbers, the resultant implant is barely at the 3D range and clinical translation is unimaginable, considering the associated three surgeries, prolonged patient distress, and prohibitively high healthcare expenditure. It is therefore an object of the present invention to develop technologies that will allow for full exploitation of the in vitro organogenesis concept.

Electrospinning produces 3D fibrous constructs that closely imitate native tissues architectural features and the high porosity of the resultant scaffolds allows for appropriate nutrient, waste and oxygen transport. Although electrospinning of temperature-responsive polymers has enabled the development of scaffold-free cell layer a commercially and clinically viable tissue-like surrogate is still onerous. This may be attributed to the large numbers of cells still required and the prolonged time in culture needed for the cells to deposit sufficient ECM that is associated with cell phenotype losses. Considering that ECM is key modulator of cell fate, strategies that integrate enhanced and accelerated ECM synthesis and deposition in the developmental cycle of in vitro organogenesis concepts may be able to bridge the gap between positive therapeutic clinical efficacy and market success.

Although various in vitro microenvironment modulators have been assessed over the years as a means to control cell fate in culture, only marginally (e.g. growth factor supplementation, oxygen tension, mechanical stimulation), if at all (e.g. surface topography, substrate rigidity), enhance and accelerate ECM synthesis and deposition. To this end, the concept of macromolecular crowding (MMC) has been introduced that, following the principles of excluded volume effect, accelerates biochemical reactions and biological processes by several orders of magnitude. Although MMC, by imitating the localised density of native tissues in vitro, has been shown to induce an up to 120-fold increase in collagen and associated ECM deposition within 4-6 days in differentiated and stem cell cultures, its safety and efficacy in regenerative medicine has yet to be assessed.

### MMC in Tissue Engineering and Regenerative Medicine

Cell-based therapies are based on the notion that tissue repair and regeneration can be accomplished best by recruiting the cells' inherent proficiency to create their own tissue-specific ECM with a precision and stoichiometric efficiency still unmatched by man-made devices. Cell injections have shown varied therapeutic efficiency, as the mode of administration offers little control over localisation, retention and distribution of the injected cell suspensions. To this end, scaffold and scaffold-free living substitutes have been developed and their therapeutic efficacy and efficiency have been demonstrated clinically for various indications (e.g. skin, cornea, blood vessel). This success has been attributed to the secreted, intertwined network of deposited ECM, which increases cell survival rate by protecting them. The ECM also acts as a biological glue, enabling localised delivery of the cells and their secretome, which is rich in bioactive and trophic factors. Despite these positive outcomes, only a handful of products have been commercialised (e.g. MACI® for cartilage and Epicel® for deep dermal/full thickness burns from Vericel; Affinity®, Apligraf® and Dermagraft® for acute and chronic wounds from Organogenesis Inc.). This limited technology transfer from bench-top to clinic has been attributed to the prolonged time required to develop an implantable device ex vivo (e.g. 14-21 days for corneal epithelium, 25-50 days for skin, 196 days for blood vessel), which is often associated with cell phenotype loss and senescence. It has been proposed that methods that enhance and accelerate native ECM synthesis and deposition in three-dimensional fashion must be integrated into the developmental cycle of advanced therapy medicinal products to bridge the gap between positive therapeutic outcomes and market success.

### MMC in Drug Discovery

Current in vitro and in vivo pathophysiology models are associated with clinical trial failure, necessitating the development of more economic, reliable and clinically relevant in vitro models. Although various animal models have been used for drug screening, they are too expensive for day-to-day assays and largely fail to accurately recapitulate human disease states, progression and metastasis. Simple and economic two-dimensional cell culture models are unsuitable for clinically relevant drug development, as cells grown in two-dimensional cultures rapidly lose their phenotype and function, cell genetic and epigenetic drift is frequently reported, paracrine signalling cascades are not initiated and, due to the very low ECM presence, cell-ECM interactions are not imitated. The latter is of significant important, as life-threating pathological conditions arise when ECM remodelling becomes excessive or uncontrolled. Fibrosis, for example, is associated with dysfunctional connective tissue metabolism, activated fibroblasts and excessive ECM production, whilst cancer is characterised by increased deregulated ECM deposition that promotes cellular transformation and metastasis. Thus, approaches that recapitulate the native three-dimensional architecture and composition of the diseased tissue must be adopted for the development of in vitro pathophysiology models.

### MMC in Cellular Agriculture Aquaculture

Cellular agriculture is an emerging technology that enables production of agricultural products, primarily meat, using cells. It has been labelled a ‘revolutionary technology’, considering the many benefits for humans, animals, the environment and society. For the successful production of cultured meat, an orchestrated, multifactorial and complementary approach is proposed that addresses the main limitations of the state-of-the-art. Specifically: Maximise muscle, fat and connective tissue deposition from respective cells in low serum or serum-free media; and utilise a scaffold of large surface area (to allow cell attachment and growth), which is stretchable (to stimulate differentiation), flexible (to allow contraction), anisotropic (to induce physiological myotube formation) and removable (to avoid toxicity).

As discussed above, macromolecular crowding is an emerging technology in the field of cell-based products that dramatically enhances and accelerates ECM deposition. However it has only been used to produce tissues in the form of sheets of cells and not three dimensional structures. In the quest of the ideal crowding molecule, sulphated polysaccharides, non-sulphated polysaccharides and synthetic polymers have been used. Despite the significant strides that have been achieved in the field, the optimal crowding molecule remains elusive, with respect to maintaining cellular phenotype and accelerating ECM deposition. Sulphated polysaccharides, non-sulphated polysaccharides and synthetic polymers are used extensively as crowding agents. Sulphated polysaccharides, due to their polydispersity and negative charge as crowding molecules for enhanced and accelerated ECM deposition. However, sulphated polysaccharides and non-sulphated polysaccharides, due to their affinity to growth factors for example, direct stem cells towards a specific lineage. Polymeric crowders, although maintaining stem cell phenotype, as they do not have affinity to growth factors, are not as effective as sulphated polysaccharides in enhancing and accelerating ECM deposition. In particular, acidic polysaccharides, due to their potential to change the pH of culture media, have been excluded as crowding molecules.

### MMC in Cell Culture Technologies

In cell culture technology, which is significant for regenerative medicine, drug discovery and the cellular agriculture/aquaculture sectors, macromolecular crowding can be used in the development and validation of specialised media and in the development of specialised substrates for effective cell expansion/lineage commitment. For example, non-sulphated crowders (e.g. Ficoll™ 70 kDa/400 kDa cocktail [1] can be used to induced adipogenesis and sulphated crowders (e.g. dextran sulphate 500 kDa [2]; carrageenan [3]; galactofucan, ulvan and fucoidan [4]) can be used to induce osteogenesis and chondrogenesis. Macromolecular crowding has also been used to assess the effectiveness of commercially available media to maintain chondrocyte phenotype in culture [5]. In the cell culture substrate development, macromolecular crowding can be used to significantly improve the efficiency of cell-derived matrices. For example, macromolecular crowding derived matrices were able to promote pigmentation in human retinal pigment epithelial cells and induce retinal pigment epithelial differentiation from pluripotent stem cells[6] and maintain phenotype and function of hematopoietic stem and progenitor cells, keratinocytes, podocytes and H9 human embryonic stem cells for over 20 passages.

EP2718421A1 describes a method for the rapid production of host-specific tissues to be used for any tissue engineering application. Using host-specific cells avoids immune rejection problems from implantation of materials from other subjects. This invention discloses the steps of culturing host cells in the presence of large poly-dispersed, negatively or neutrally charged macromolecular crowders to produce tissue substitutes for human tissue engineering.

Lareu et al. (Febs Letters vol 581, no. 14, pgs 2709-2714) describes the excluded volume effect (EVE), a process that occurs as a result of using macromolecules to occupy a given space and thereby accelerate the reaction kinetics of other molecules in the remaining space. It was shown that implementing the EVE approach in fibrobrast culture accelerated conversion of procollagen to collagen. Consequently, this resulted in a 20-30 fold increase in collagen deposition in 2-dimensional cultures, and 3-6 fold increased collagen deposition in 3-dimensional cultures.

KR20200025616A discloses a method for manufacturing three-dimensional cell culture scaffolds using a biocompatible polymer and a temperature-sensitive polymer. The temperature-sensitive polymer has a grid-like structure which forms the base for the cell culture portion. The spheroids cultured on these scaffolds can be easily recovered by controlling the temperature of the cell culture.

It is thus an object of the invention to develop a truly 3D tissue substitute, using only a fraction of the cells and time that traditional in vitro organogenesis approaches utilise. A further object is to produce a therapeutically efficacious and safe truly 3D tissue substitute.

It is a further object of the present invention to provide a process for the production of 3D tissue substitutes or artificial tissue constructs which is rapid. In particular the object of the invention is to provide a method of producing commercially viable quantities of tissue substitutes within a period of days, as opposed to weeks or months that traditional methods require.

In particular it is an object of the invention that the substitute can be produced within about 2 to 14 days. Another object is to provide 3D tissue surrogates (cellular and acellular) and methods of producing them, the tissues being useful for tissue engineering, drug discovery, cellular agriculture/aquaculture, cell culture technologies and biomedicine applications. Such applications include: Tendon regeneration, Bone regeneration, Nerve regeneration, Cornea regeneration, Skin regeneration etc; Drug delivery, drug discovery, gene delivery, gene discovery; In vitro systems (e.g. development of cancer therapeutics; development blood-brain barrier systems, fibrosis models; cancer models, etc.); Coatings of medical devices to avoid immune response, cell expansion substrates, tissue glues/adhesives, improvement of processes; Meat and fish products for food and animal consumption.

A further object of the invention is to provide an additional macromolecular crowder. Such a crowder may have improved properties compared to existing crowders. A still further object is to provide use of polyacrylic acid as a macromolecular crowder to enhance and/or accelerate ECM deposition. In particular, it is an object to develop a tissue substitute using polyacrylic acid as a molecular crowder.

### SUMMARY OF THE INVENTION

According to the present invention there is provided a method for the production of a three-dimensional (3D) tissue substitute or surrogate comprising culturing cells in the presence of a three-dimensional scaffold and one or more macromolecular crowders, wherein the macromolecular crowders are generally large poly-dispersed macromolecules. Two or more macromolecular crowders may be preferred.

The scaffold may be a sponge, an electrospun scaffold, a hydrogel or the like. The scaffold may be a ceramic, a synthetic polymer or a natural polymer. Ceramic scaffolds generally comprise hydroxyapatite (HA) and/or tri-calcium phosphate (TCP). Synthetic polymers generally include polystyrene, poly-l-lactic acid (PLLA), polyglycolic acid (PGA) and poly-dl-lactic-co-glycolic acid (PLGA). Natural polymers generally include collagen, hyaluronic acid, various proteoglycans, alginate-based substrates and chitosan. The collagen may comprise or consist of Type 1 colagen

In any embodiment, the scaffold is a temperature-sensitive copolymer fibre scaffold for the development of scaffold-free tissue substitutes.

In any embodiment, the scaffold is produced by electrospinning. Electrospun scaffolds are typically very dense, and cell/ECM penetration has not been possible using conventional scaffold fabrication processes. In the present invention electrospinning was used as proof of concept, as it is the least porous/most dense scaffold so if the process works with it, it will work with any other scaffold.

In any embodiment, the scaffold is produced by freezing or freeze-drying (lyophilisation).

Temperature sensitive copolymer fibres, also known as temperature-responsive polymers or thermoresponsive polymers, are polymers that exhibit a drastic and discontinuous change of their physical properties with temperature. In the present invention the temperature sensitive polymer will dissolve with a change of temperature shift leaving cells formed as a three-dimensional structure behind. Suitably the copolymers are sensitive to a temperature shift of from about 37° C. to about 4° C., but the temperature shift required is specific to the polymer used.

Suitable temperature sensitive copolymers include Poly-N-isopropylacrylamide-N-tert-butylacrylamide (pNIPAM-NTBA) copolymers, hydroxybutyl chitosan, poly(N-isopropyl-acrylamide) and its copolymers. The macromolecules may be negatively charged or neutral macromolecules. The large poly-dispersed macromolecules may be selected from the group comprising of: synthetic polymers (e.g. polyethylene glycol, polyvinylpyrrolidone, polysodium-4-styrene sulfonate, polyvinyl alcohol, polyacrylic acid, etc), natural polysaccharides (e.g. carrageenan; high, low and non-sulphated dextran; Ficoll™, gums, such as gum Arabic, gum gellan, gum karaya, gum xanthan, etc) and glycosaminoglycans (e.g. heparin, heparin sulphate, hyaluronic acid, etc), alone or in cocktail. Particularly preferred is carrageenan. Carrageenan may be used in combination with one or more of the molecules as defined above.

Carrageenans are a family of linear sulphated polysaccharides extracted from red seaweeds. There are several types of carrageenan: Kappa, lambda and iota, all of which would be suitable for use in this invention, as would combinations or blends thereof. Particularly preferred is a mixture of Kappa and Lambda carrageenan (available from Sigma-Aldrich). Kappa or Lambda carrageenan may also be used individually (e.g. lambda medium viscosity, provided by IMCD UK Limited).

Gums are also preferred examples of large poly-dispersed macromolecules. Particularly preferred examples include gum Arabic, gum gellan, gum karaya, and gum xanthan. Any of these gums may be used individually or as a combination. Any of these gums may also be used in combination with one or more of the molecules as defined above.

In any embodiment, the macromolecular crowder may be used at a level of between about 1 μg/ml and about 500 mg/ml, for example 10 μg/ml to 500 μg/ml, 100 μg/ml to 500 μg/ml, 1 μg/ml to 100 μg/ml, 10 μg/ml to 90 μg/ml, 20 μg/ml to 80 μg/ml, 30 μg/ml to 70 μg/ml, 40 μg/ml to 60 μg/ml or about 50 μg/ml, with the amount used depending on the physicochemical properties (e.g. concentration, dispersity, size, shape, charge, molecular weight, etc) of the crowder/crowding cocktail.

Any cell types may be used in the invention. The cells may be selected from permanently differentiated cells (e.g. skin, tendon, cornea, lung, breast fibroblasts; osteoblasts; chondrocytes), or stem cells (e.g. bone marrow, adipose-derived, umbilical cord, etc). The cells may be engineered. In any embodiment, the cells are or are derived from human cells.

The cells may be cultured in the presence of culture medium supplemented with a serum or serum, for example fetal bovine serum, human serum, porcine serum, chicken serum, ascorbic acid phosphate, or a combination thereof. The serum may be used at 0.1% to 40% volume to volume. Suitable concentrations of serum include, 0.5% to 30%, and 5% to 20%. Alternatively the cells may be cultured with no serum present.

The invention also provides a 3D tissue substitute or surrogate produced according to a method of the invention. Using aligned fibres, it is possible to make aligned tissues for tendon, skin, cornea repair for example. With tubular scaffolds, it is possible to make tubular tissues for blood vessel, peripheral nerve, etc. Using porous scaffolds, tissue for cartilage and bone, etc can be made.

As used herein the term “molecules” includes molecules, spheres, particles and polymers. Suitable molecules are disclosed in EP 2 718 421. As used herein the term “poly-dispersed” means that the molecules have a broad range of size, shape and mass characteristics, as opposed to molecules which have a uniform size, shape and mass distribution which are mono-dispersed molecules. Polymer materials are poly-dispersed if their chain length varies over a wide range of molecular masses. It would be possible to increase the polydispersity of the crowder, by using a combination of two or more crowders. For example, a mixture of carrageenan and dextran sulphate would be more poly-dispersed than carrageenan alone.

The invention also provides a 3D tissue substitute or surrogate according to the invention or produced according to a method of the invention, for use in a method of treating a wound or scar in a mammal, in which the 3D tissue substitute or surrogate is applied to the wound or scar. In any embodiment, the wound is a topical wound. |The method may be to improve wound healing (e.g. to provide better or faster wound healing, or reduced scar index). In any embodiment, the scaffold comprises or consists essentially of collagen, typically Type 1 collagen. In any embodiment, the scaffold comprises bone marrow stem cells. In any embodiment, the 3D tissue substitute or surrogate has a planar shape. The 3D tissue substitute or surrogate according to the invention or produced according to a method of the invention may also be employed in other tissue engineering applications including but not limited to nerve cell regeneration and tissue membrane regeneration.

One aspect of the invention enables the production of a tissue substitute of more than 300 μm in thickness within about 10 days whilst the prior art systems take up to 28 days to produce a thickness of only 10 to 50 μm. One aspect of the system of the invention utilises a fraction of cells that customary approaches use (for example, 50 K cells per cm², whilst the prior art methods use over 500 K cells per cm²).

Also provided is a process for the production of a tissue comprising culturing cells in the presence of one or more macromolecular crowders, optionally wherein at least one of the macromolecular crowders is polyacrylic acid.

In any embodiment, the polyacrylic acid may have an average molecular weight of from about 400 kDa to about 5000 kDa, for example 450 to 1000 kDa, 1000 kDa to 5000 kDa, 1000 kDa to 4000 kDa, 1000 kDa to 3000 kDa, 1000 kDa to 2000 kDa, 2000 kDa to 3000 kDa, 3000 kDa to 4000 kDa, 4000 kDa to 5000 kDa, 450 kDa, 1000 kDa, or 4000 kDa.

In any embodiment, the cells are cultured in the presence of a three-dimensional scaffold. In any embodiment, the scaffold may be a ceramic, a synthetic polymer or a natural polymer as described above. Preferably the scaffold is a temperature-sensitive copolymer fibre scaffold. Preferably the scaffold is produced by electrospinning.

The one or more molecular crowders may further comprise one or more of the following: synthetic polymers (e.g. polyethylene glycol, polyvinylpyrrolidone, polysodium-4-styrene sulfonate, polyvinyl alcohol, etc), natural polysaccharides (e.g. carrageenan; high, low and non-sulphated dextran; Ficoll™, gums, etc) and glycosaminoglycans (e.g. heparin, heparin sulphate, hyaluronic acid, etc), and combinations or blends thereof.

The polyacrylic acid may be used in an amount of from about 1 μg/ml culture medium to about 50,000 μg/ml culture medium, for example 10 μg/ml, 50 μg/ml, 100 μg/ml, 500 μg/ml, 1000 μg/ml, 5000 μg/ml, 10,000 μg/ml, or 50,000 μg/ml.

Despite causing a decrease in pH of the culture medium used, the use of polyacrylic acid as a macromolecular crowder surprisingly enhances and accelerates ECM deposition at similar rates to carrageenan, which is the most effective crowder that has been used to date.

Also provided herein is a substitute or surrogate tissue, for example a tissue sheet or a 3D tissue, produced by the method described above. Further provided herein is the use of polyacrylic acid as a macromolecular crowder for tissue production.

As used herein the term scaffold generally means a highly porous biomaterial which acts as template for tissue regeneration, to guide the growth of new tissue. The scaffolds are often three dimensional to provide the appropriate environment for the regeneration of tissues and organs, and are typically seeded with cells and occasionally growth factors, or subjected to biophysical stimuli. These cell-seeded scaffolds are then either cultured in vitro to synthesize tissues which can then be implanted into an injured site, or are implanted directly into the injured site, where regeneration of tissues or organs is induced in vivo.

### BRIEF DESCRIPTION OF THE DRAWINGS

### 3D Tissue Production Using Large-Polydispersed Macromolecules as Macromolecular Crowders

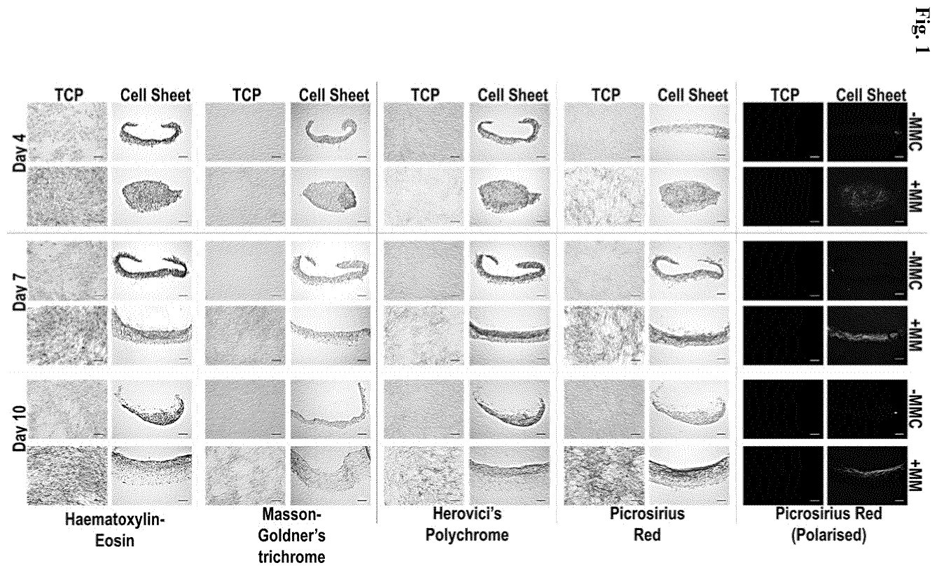

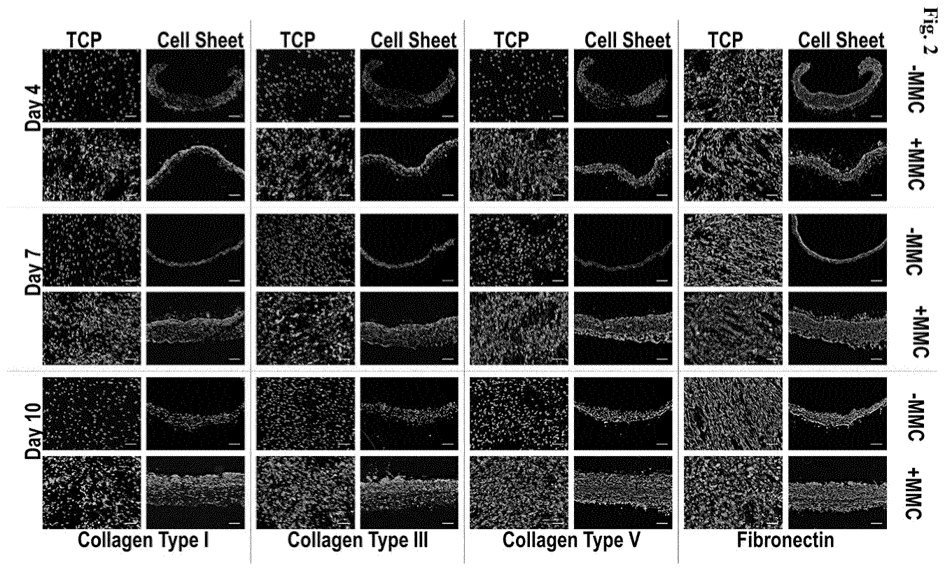

FIG. 1: Histological analysis of hADSCs grown without (−) and with (+) MMC on two-dimensional TCP revealed that MMC increased ECM deposition (pink in haematoxylin-eosin), which was primarily collagenous (green in Masson-Goldner's trichrome and bright red in Picrosirius red) and was maturing as a function of time in culture (young collagen blue and mature collagen pink to red in Herovici's polychrome), but did not improve structural order (no signal in polarised microscopy). Histological analysis of hADSCs grown without (−) and with (+) MMC on three-dimensional temperature-responsive electrospun scaffolds (the samples were obtained after dissolving the scaffolds by switching the temperature) revealed similar results, but now MMC allowed the development of a three-dimensional tissue-like structure and, importantly, polarised microscopy (stained bright red) made apparent that MMC induced a high organisational order. Scale bar: 100 μm. N=3.

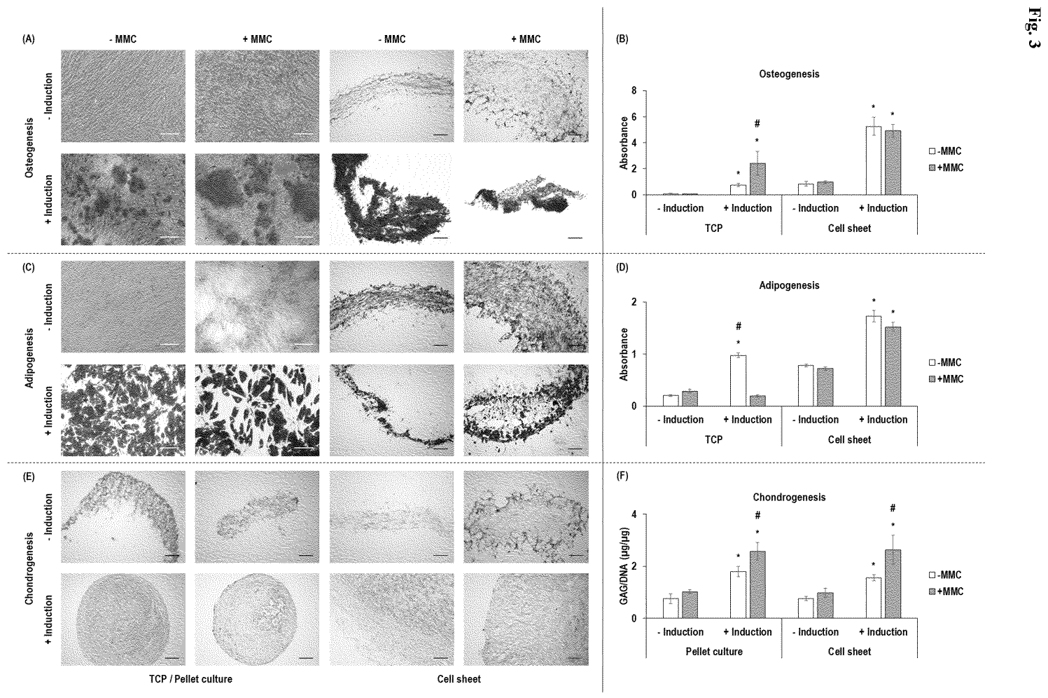

FIG. 2: Immunocytochemistry analysis of hADSCs grown without (−) and with (+) MMC on two-dimensional TCP revealed that MMC increased collagen type I (green), collagen type III (green) and collagen type V (green) deposition, without affecting fibronectin (green) deposition. Histological analysis of hADSCs grown without (−) and with (+) MMC on three-dimensional temperature-responsive electrospun scaffolds (the samples were obtained after dissolving the scaffolds by switching the temperature) revealed similar results, but now MMC allowed the development of a three-dimensional tissue-like structure. Nuclei: blue. Scale bar: 100 μm. N=3.

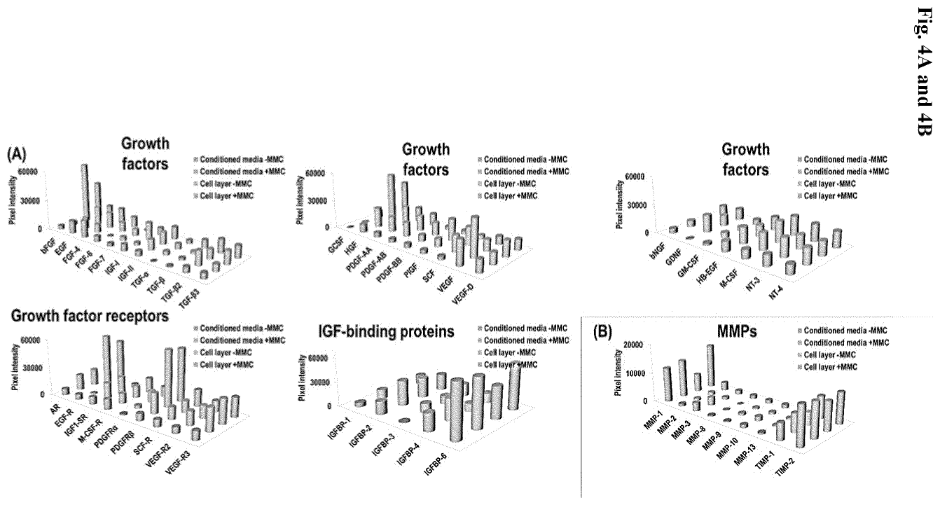

FIG. 3: Osteogenic differentiation (A) and absorbance quantification of Alizarin red staining (B) revealed that MMC induced significantly (p<0.05) higher amounts of calcium nodules of hADSCs grown on two-dimensional TCP, whilst no significant (p>0.05) differences were observed between without (−) and with (+) MMC when hADSCs were grown on three-dimensional temperature-responsive electrospun scaffolds (the samples were obtained after dissolving the scaffolds by switching the temperature). Adipogenic differentiation (C) and absorbance quantification of oil red O staining (D) revealed that MMC significantly (p<0.05) reduced the adipogenic potential of hADSCs grown on two-dimensional TCP, whilst no significant (p>0.05) differences were observed between without (−) and with (+) MMC when hADSCs were grown on three-dimensional temperature-responsive electrospun scaffolds (the samples were obtained after dissolving the scaffolds by switching the temperature). Chondrogenic differentiation (E) and GAG quantification analysis (F) revealed that MMC significantly (p<0.05) increased GAG content of hADSCs that were grown on both three-dimensional pellet culture and three-dimensional temperature-responsive electrospun scaffolds (the samples were obtained after dissolving the scaffolds by switching the temperature). * indicates statistically significant difference to the corresponding-induction control group (p<0.05). # indicates statistically significant difference to −MMC (p<0.05). Scale bar 100 μm. N=3.

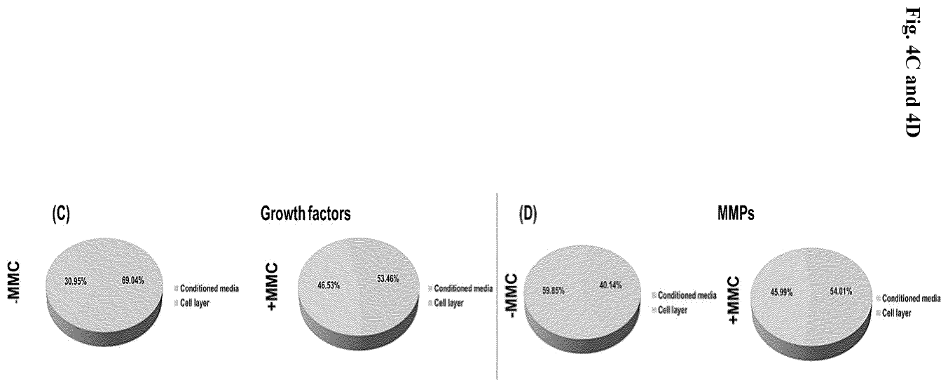

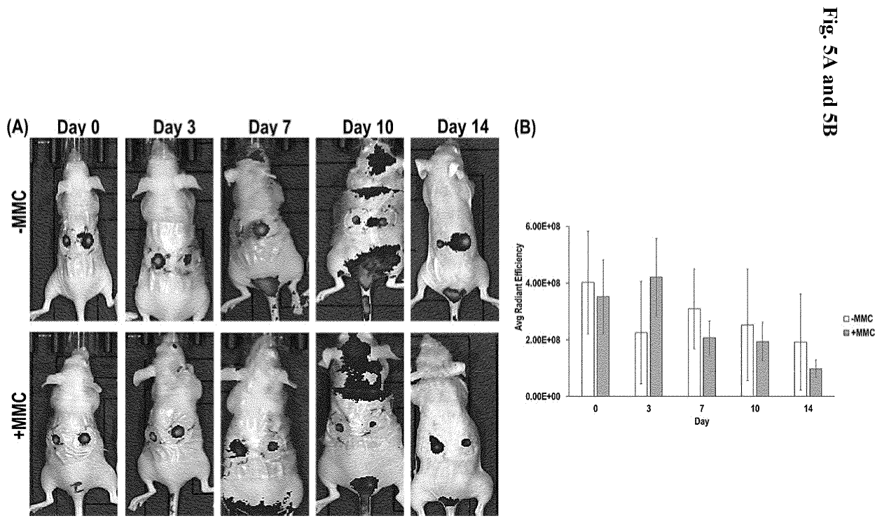

FIG. 4: Growth factor (A) and MMPs (B) antibody array quantification analyses and ratio between soluble and matrix-bound growth factors (C) and MMPs (D) of hADSCs cultured on 85:15 pNIPAM-NTBA electrospun scaffolds revealed that MMC increased growth factor content in the conditioned media and increased MMP content in the cell layers.

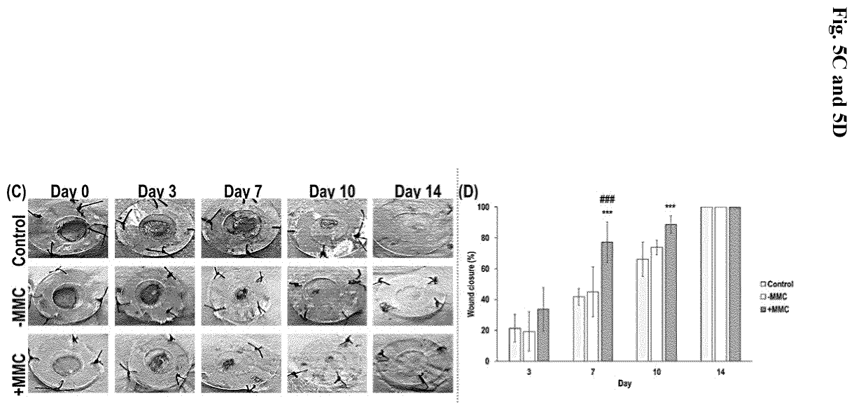

FIG. 5: In vivo cell tracking (A) and complementary average radiance efficiency analysis (B) revealed no significant (p>0.05) differences in hADSC retention at the site of implantation between the without (−) and with (+) MMC groups. Qualitative (C) and quantitative (D) wound closure analysis revealed that at day 7 and day 10, the MMC group induced the highest (p<0.001) % of wound closure. *** indicates statistically significant difference to the control group (p<0.001). ### indicates statistically significant difference to −MMC group (p<0.001). N=6 for both experiments.

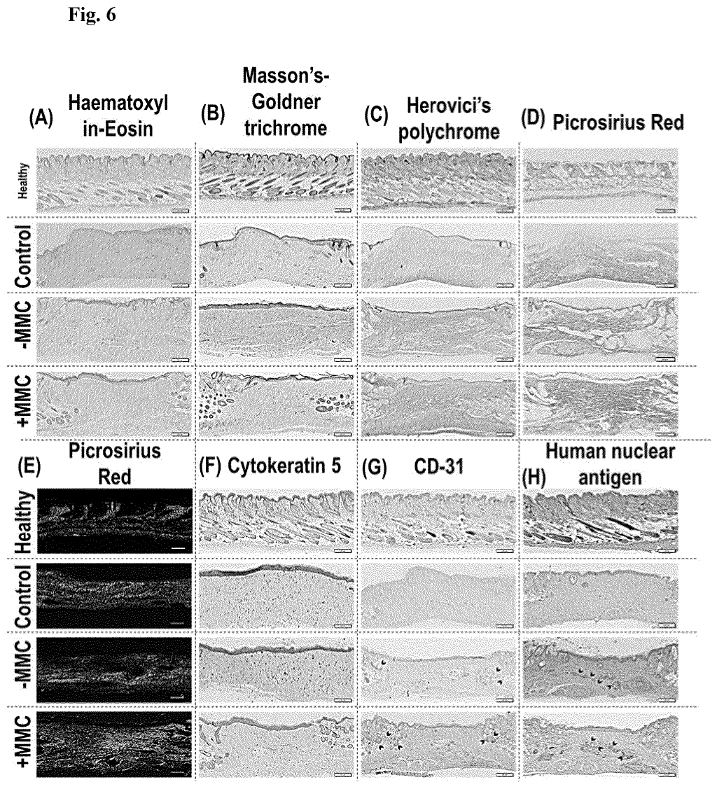

FIG. 6: (A) Haematoxylin-eosin staining showed complete re-epithelisation in all groups. (B) Masson-Goldner's trichrome staining revealed dense collagenous tissue formation in the without (−) and with (+) MMC groups, but not in the control group. (C) Herovici's polychrome staining showed that the without (−) and, in particular, the with (+) MMC groups formed neotissue composed of mature collagen, whilst the control group formed neotissue composed of immature collagen. (D) Picrosirius red staining and (E) complementary polarised microscopy revealed that the without (−) and, in particular, the with (+) MMC groups formed neotissue with ECM mainly composed of densely packed mature collagen fibres, whilst the control group formed neotissue mainly composed of loosely packed immature collagen network. (F) Immunohistochemical analysis of cytokeratin 5 showed increased number of hair follicles in the +MMC group in comparison to the −MMC and control groups. (G) Immunohistochemical analysis of CD31 positive cells showed the presence of a higher number of neo-formed blood vessels in the +MMC group in comparison to the −MMC and control groups. (H) Immunohistochemical staining of human nuclear antigen revealed that the without (−) and with (+) MMC groups retained hADSCs at the site of implantation. All images are at 14 days post-implantation. Scale bar 200 μm. N=6.

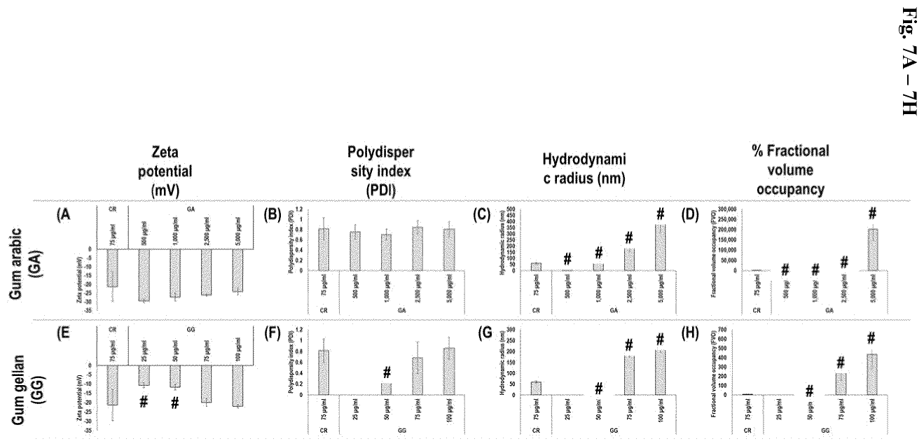

FIG. 7: The biophysical properties of hADSCs grown using four different gums as the macromolecular crowder. The gums used were gum Arabic (A-D), gum gellan (E-H), gum karaya (I-L), and gum xanthan (M-O).

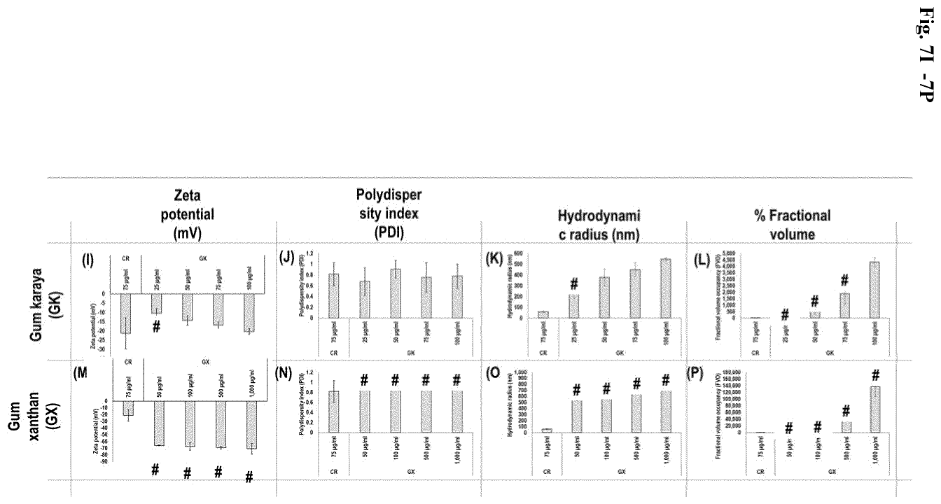

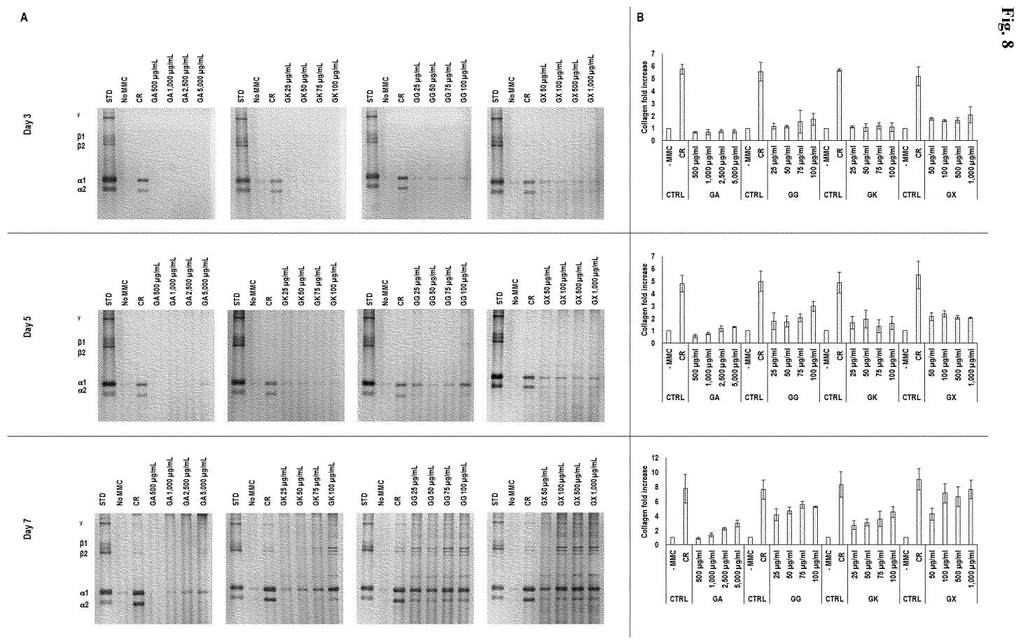

FIG. 8: SDS-PAGE (A) and collagen fold increase (B) data are shown for of hADSCs grown in the presence of gum Arabic, gum gellan, gum karaya, and gum xanthan as the molecular crowder and compared to results achieved without (−) MMC and with carrageenan. At all time points and concentrations tested both gum gellan (GG) and gum xanthan (GX) exhibited higher collagen fold increase values than the (−)MMC control. Gum karaya (GK) showed improved values over the (−) MMC control at all concentrations at day 5 and day 7. Gum Arabica (GA) showed improved values over the (−)MMC control at day 5 and day 7 with concentrations of 2500 μg/ml upwards and 1000 μg/ml upwards respectively.

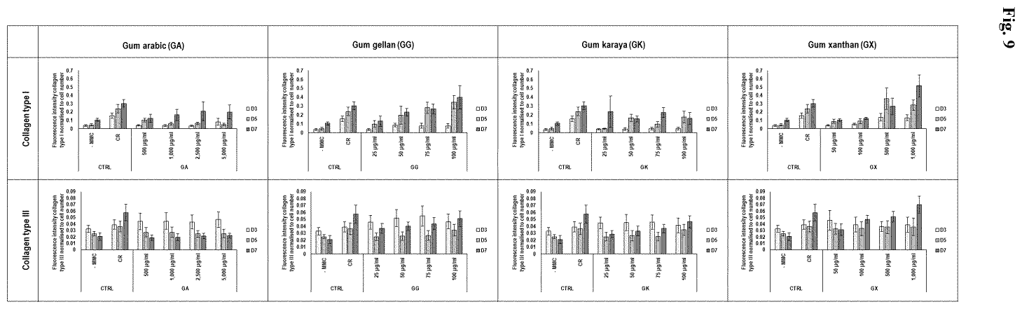

FIG. 9: Immunocytochemistry analysis was carried out on hADSCs grown in the presence of gum Arabic, gum gellan, gum karaya, and gum xanthan and compared to results achieved without (−) MMC and with carrageenan. The fluorescence intensity of collagen I and collagen III was measured and normalised to cell number.

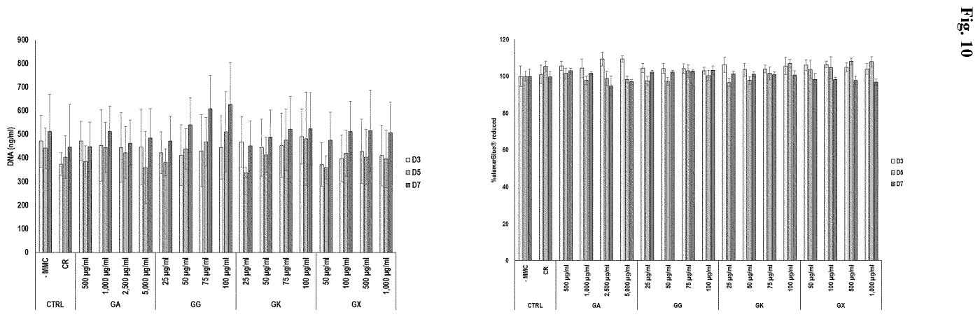

FIG. 10: DNA quantification and metabolic activity were assessed for hADSCs grown in the presence of gum Arabic, gum gellan, gum karaya, and gum xanthan and compared to results achieved without (−) MMC and with carrageenan.

Tissue Production from Human WS1 Skin Fibroblasts Using Polyacrylic Acid as a Macromolecular Crowder

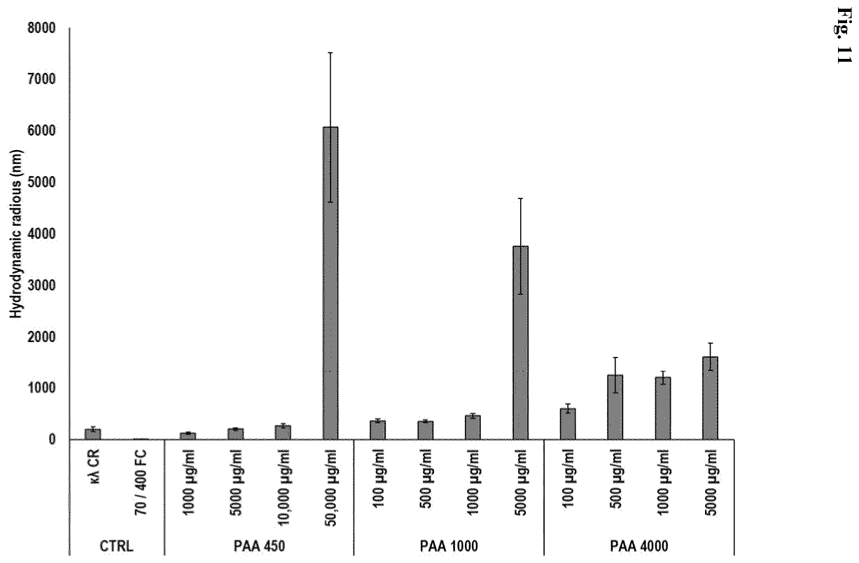

FIG. 11: Hydrodynamic radius was assessed for cells grown from human WS1 skin fibroblasts using polyacrylic acid as a macromolecular crowder.

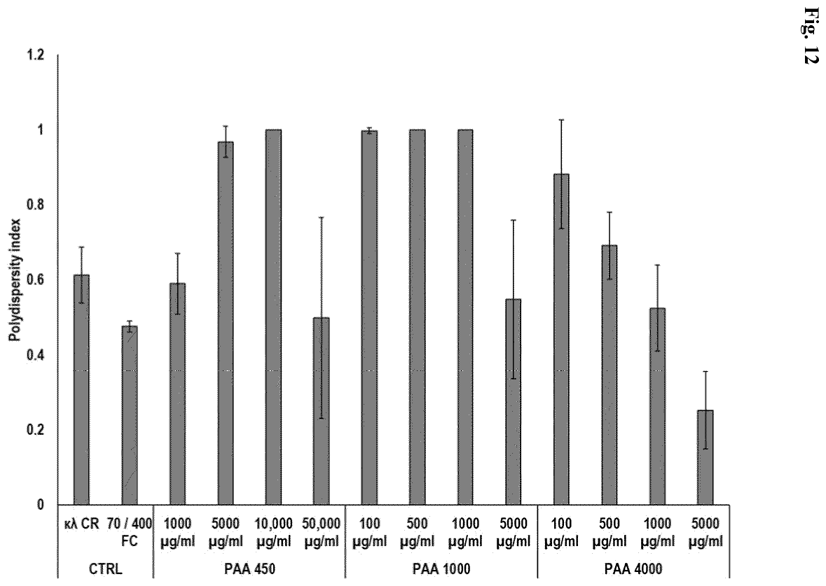

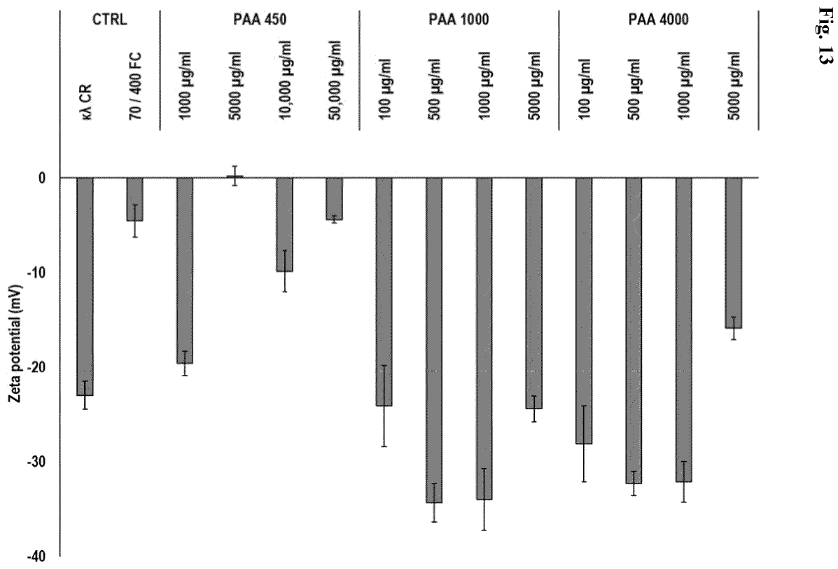

FIG. 12: Polydispersity index was assessed for cells grown from human WS1 skin fibroblasts using polyacrylic acid as a macromolecular crowder.

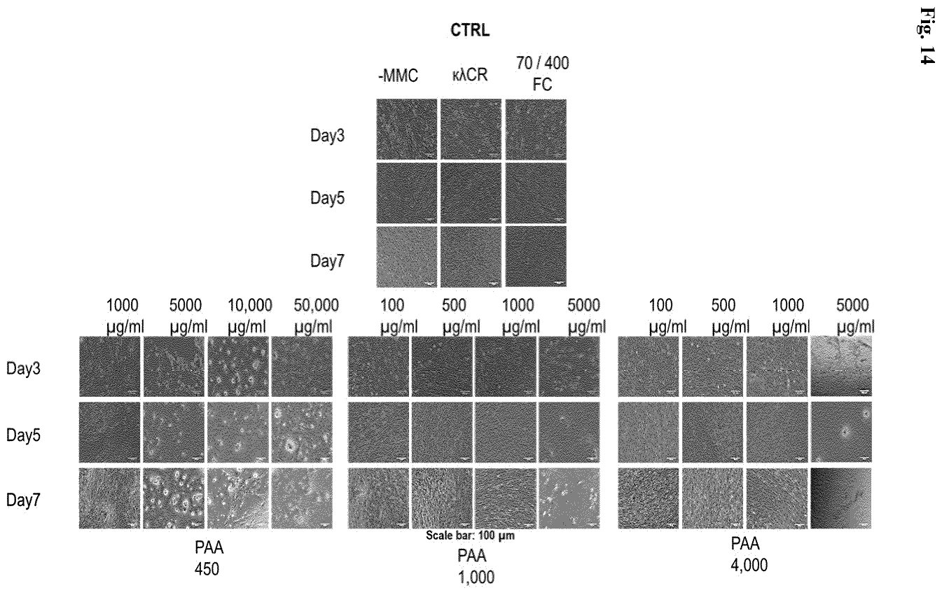

FIG. 13: Zeta potential was assessed for cells grown from human WS1 skin fibroblasts using polyacrylic acid as a macromolecular crowder.

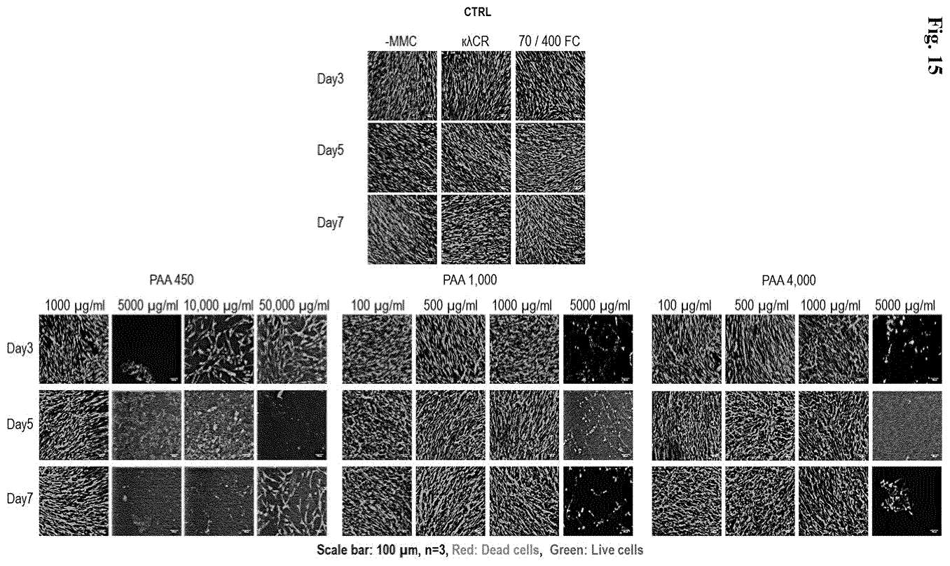

FIG. 14: Cell Morphology of cells grown from human WS1 skin fibroblasts using polyacrylic acid as a macromolecular crowder and compared to controls without (−) MMC, with carrageenan, and with 70/400 FC.

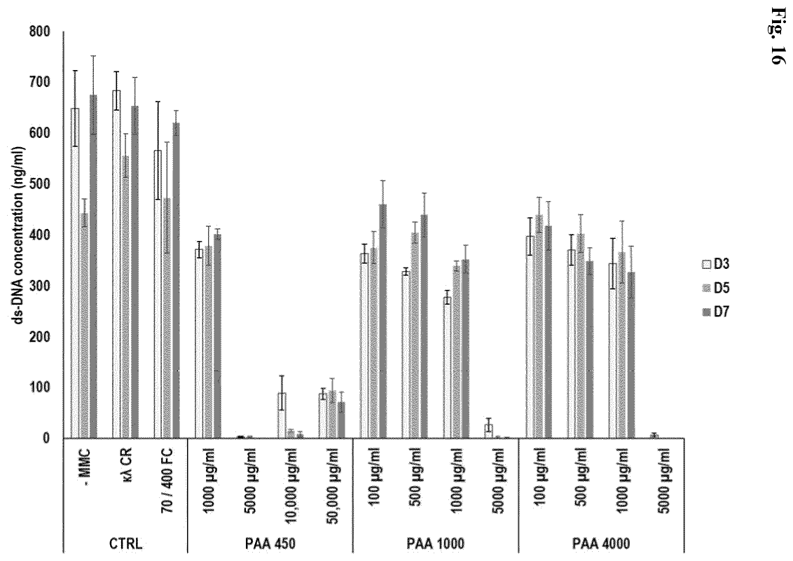

FIG. 15: Cell Viability data is shown for cells grown from human WS1 skin fibroblasts using polyacrylic acid as a macromolecular crowder and compared to controls without (−) MMC, with carrageenan, and with 70/400 FC.

FIG. 16: Cell proliferation was measured for cells grown from human WS1 skin fibroblasts using polyacrylic acid as a macromolecular crowder.

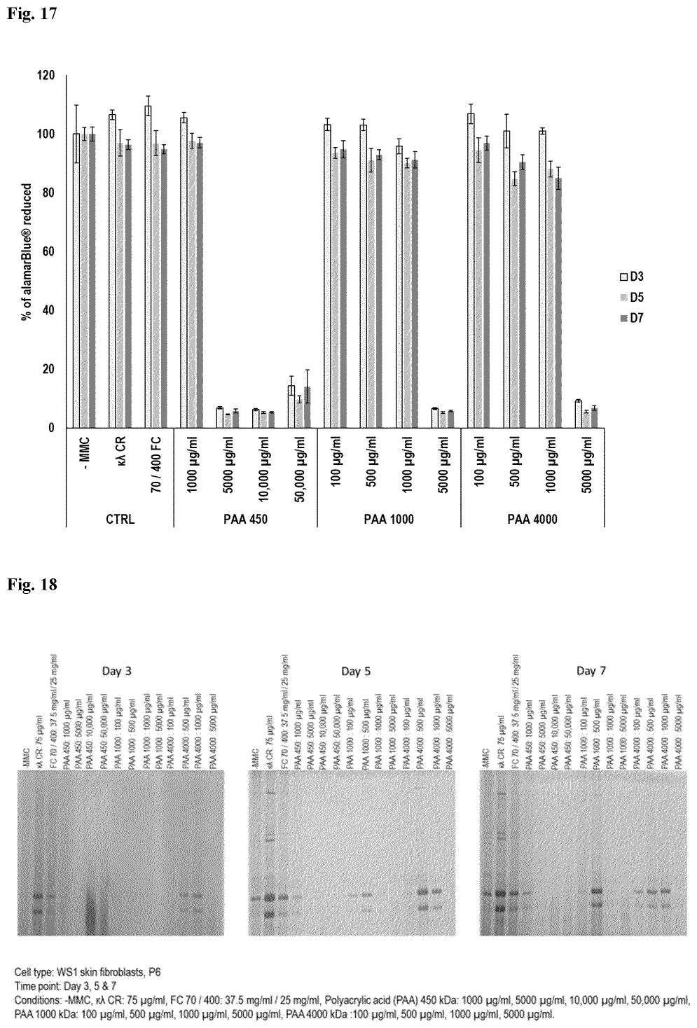

FIG. 17: Cell metabolic activity results.

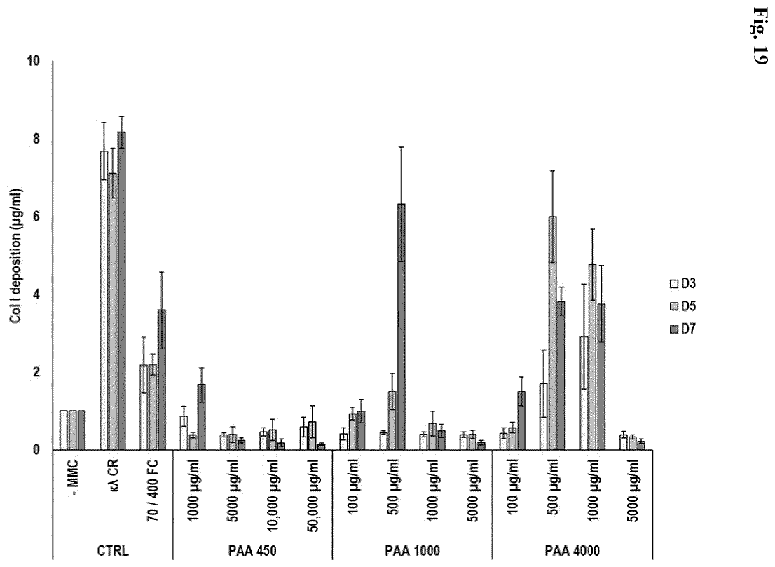

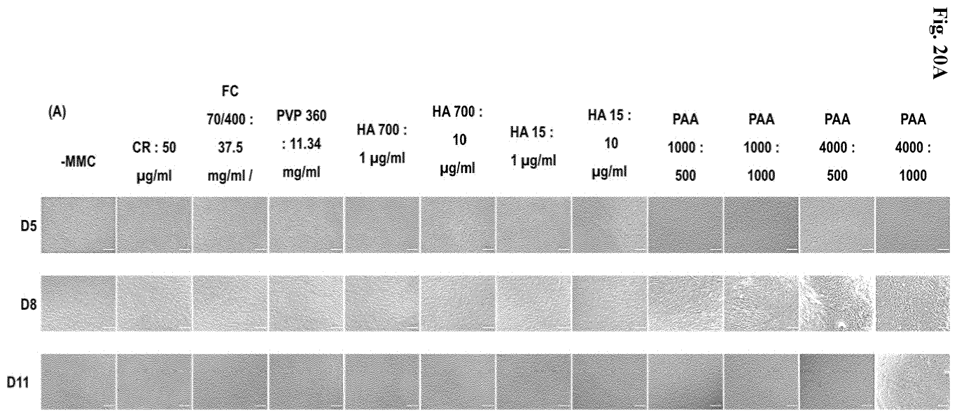

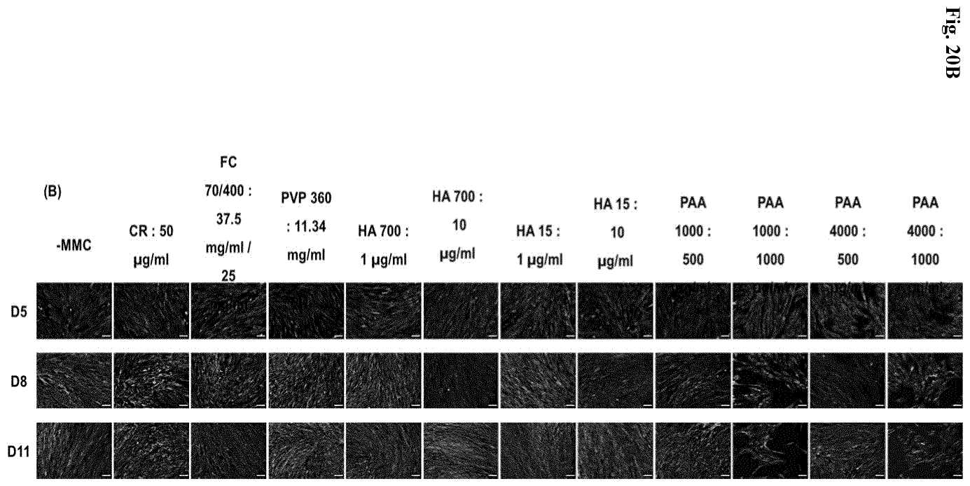

FIG. 18: SDS-PAGE data clearly illustrate that PAA enhances and accelerates ECM deposition in comparable rates to carrageenan, the most effective crowder that has been used to-date.

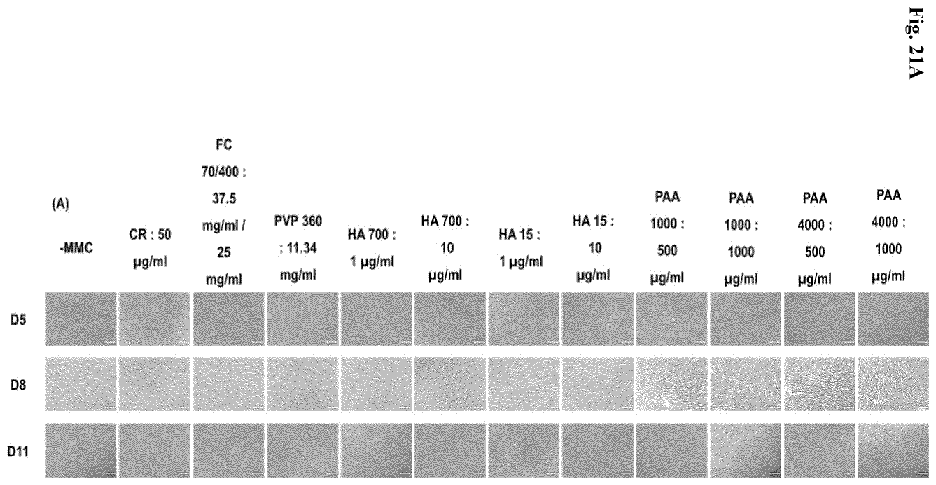

FIG. 19: Complementary densitometric analysis shows collagen I deposition achieved with PAA as a macromolecular crowder.

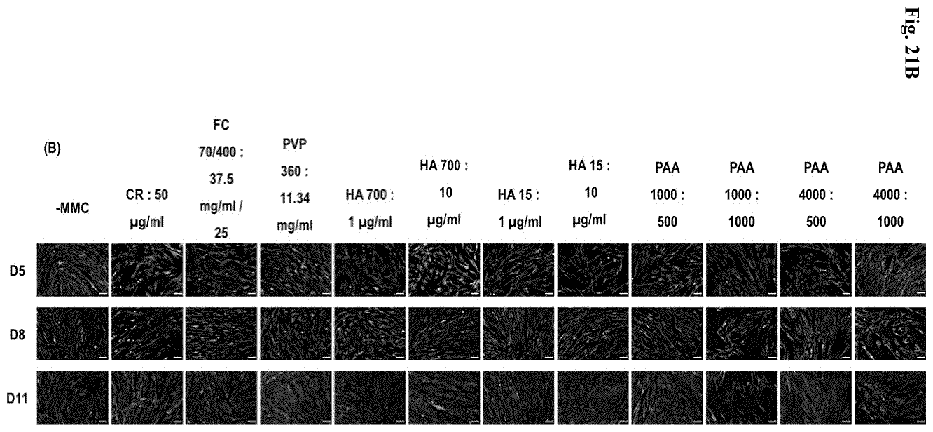

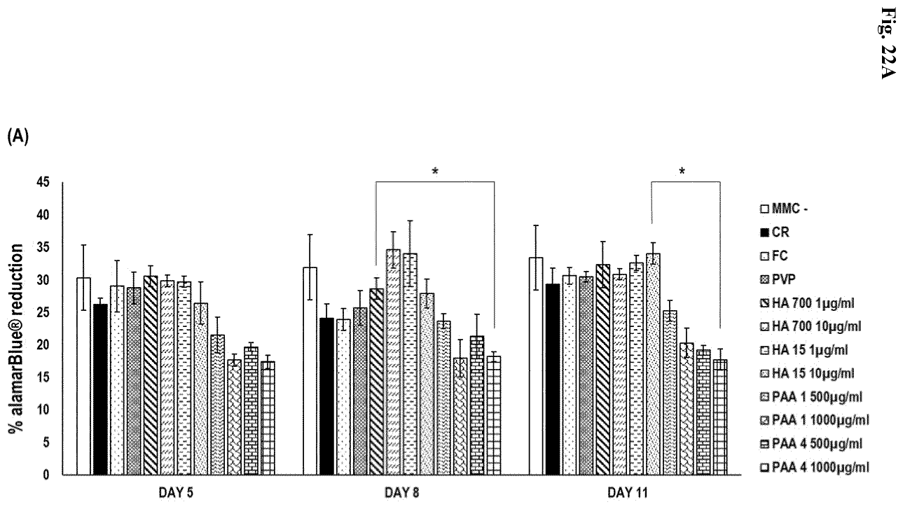

FIG. 20: Cell morphology (A) and viability (B) for Donor 1 were not affected as a function of the different crowders used. Scale bars: 100 m.

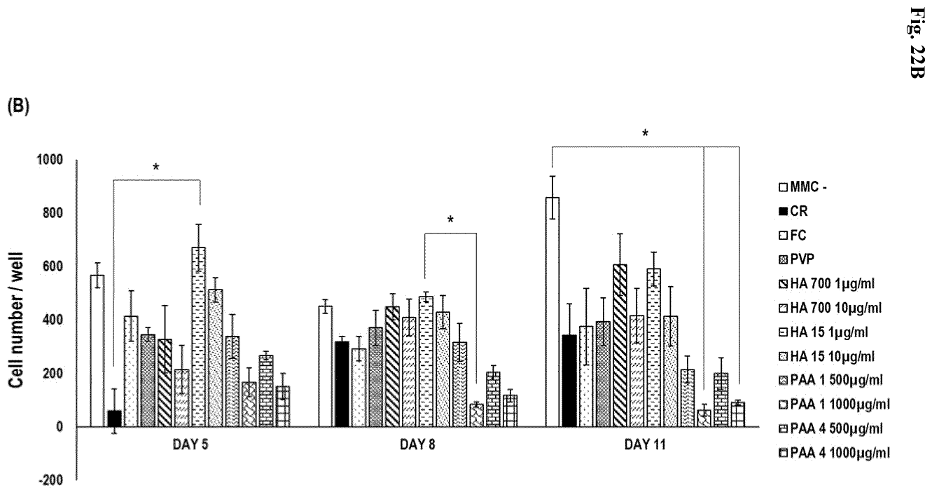

FIG. 21: Cell morphology (A) and viability (B) for Donor 2 were not affected as a function of the different crowders used. Scale bars: 100 m.

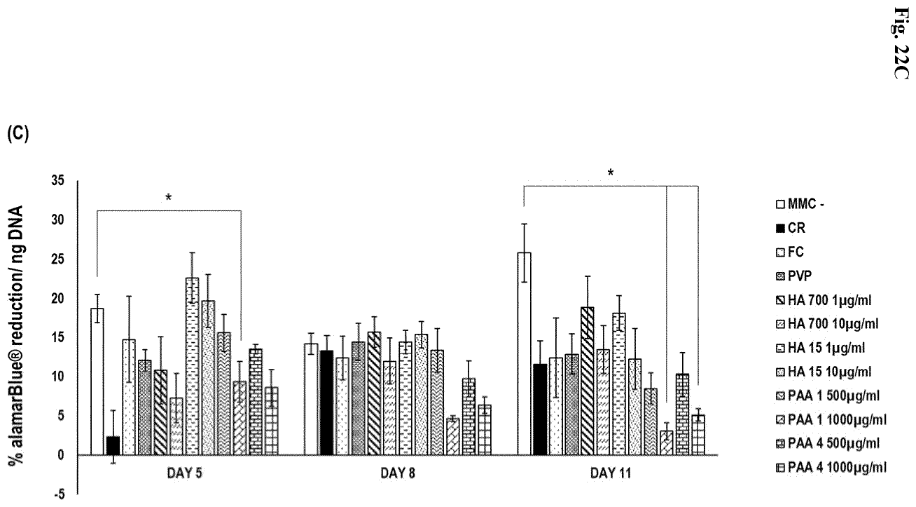

FIG. 22: Metabolic activity (A) and cell proliferation (B) for Donor 1 at day 5, 8, 11 of BM-MSCs cultured without MMC (−MMC) and in the presence of crowders. (C) Metabolic activity is expressed in terms of percentage of reduced alamarBlue™ normalised to the DNA quantity (μg/ml) obtained from the Quant-iT™ PicoGreen® dsDNA assay. * Indicates statistically significant differences p<0.05. (One-way-ANOVA test, followed by Kruskal-Wallis test).

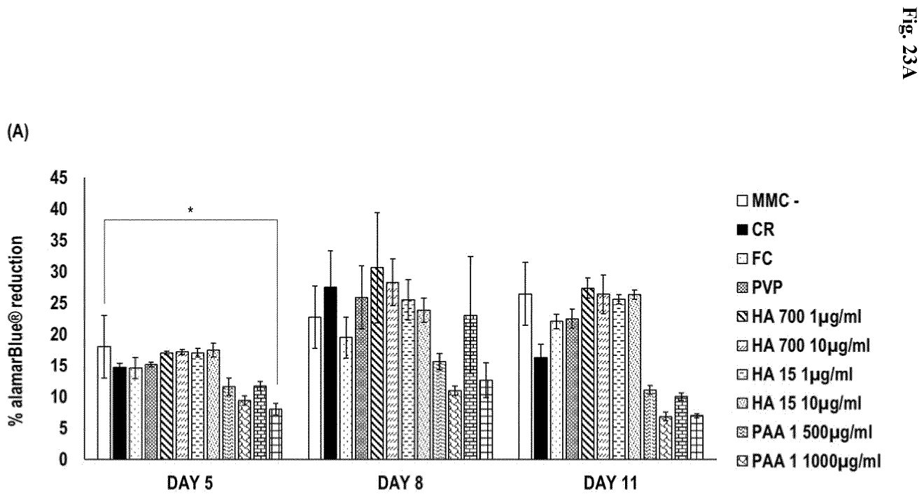

FIG. 23: Metabolic activity (A) and cell proliferation (B) for Donor 2 at day 5, 8, 11 of BM-MSCs cultured without MMC (−MMC) and in the presence of crowders. (C) Metabolic activity is expressed in terms of percentage of reduced alamarBlue™ normalised to the DNA quantity (μg/ml) obtained from the Quant-iT™ PicoGreen® dsDNA assay. * Indicates statistically significant differences p<0.05. (One-way-ANOVA test, followed by Kruskal-Wallis test).

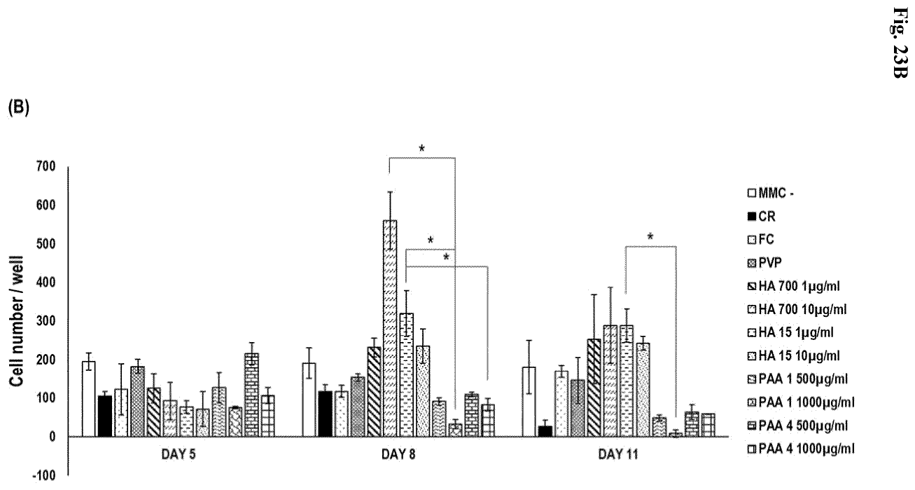

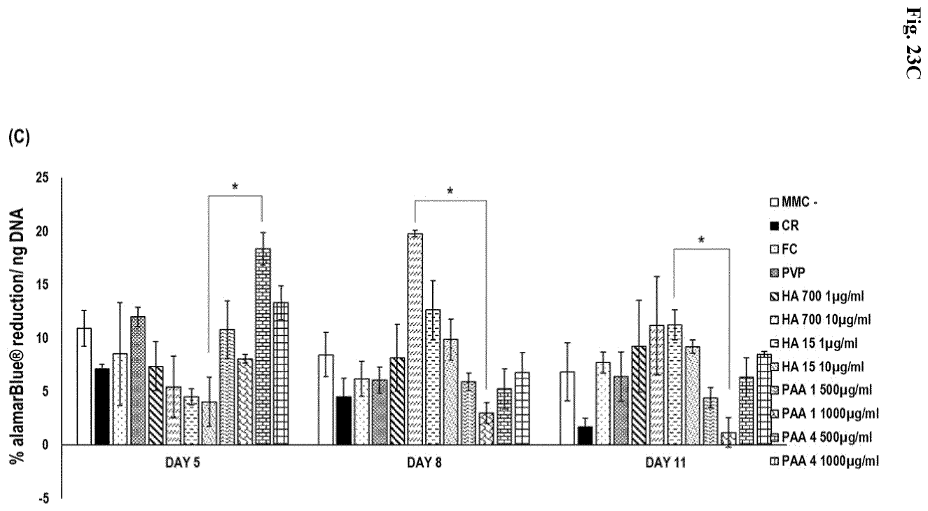

FIG. 24: SDS-PAGE (A) and complementary densitometric analysis (B) for Donor 1 revealed that at all timepoints carrageenan (CR) and PAA 4.000.000 kDa (500,1000 μg/ml) induced higher collagen I deposition compare to the control group (−MMC). * Indicates statistically significant differences p<0.05. (One-way-ANOVA test, followed by Kruskal-Wallis test).

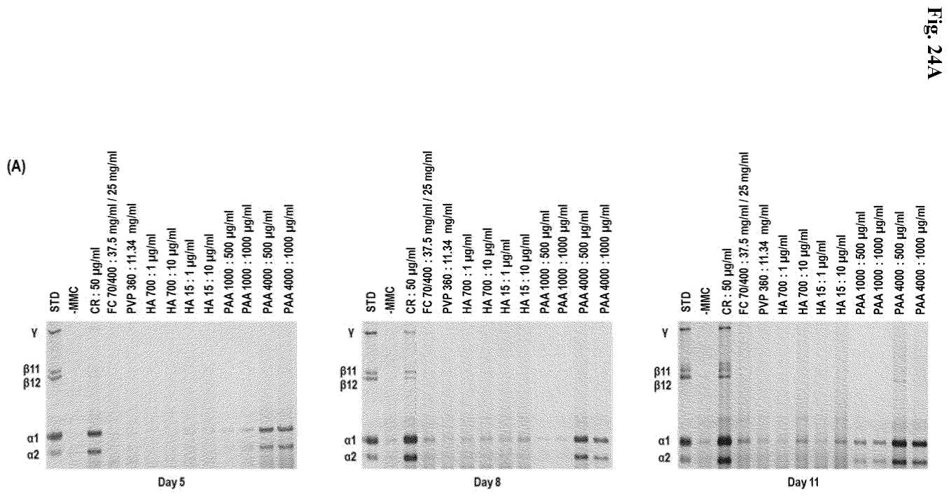

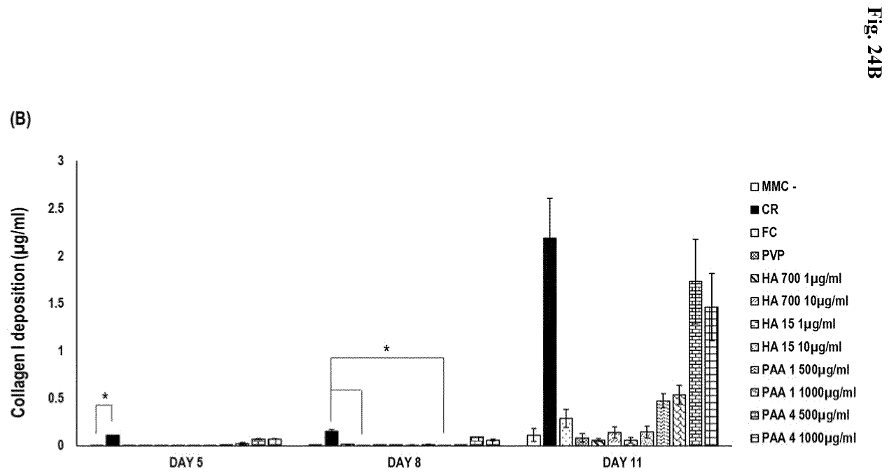

FIG. 25: SDS-PAGE (A) and complementary densitometric analysis (B) for Donor 2 revealed that at all timepoints carrageenan (CR) and PAA 4.000.000 kDa (500,1000 μg/ml) induced higher collagen I deposition compare to the control group (−MMC). * Indicates statistically significant differences p<0.05. (One-way-ANOVA test, followed by Kruskal-Wallis test) FIG. 26: Immunocytochemistry analysis of collagen type I for Donor 1 at days 5, 8 and 11 of BM-MSCs cultured without MMC (−MMC) and in the presence of crowders. Collagen type I: Green, Nuclei: Blue. Scale bars: 100 μm.

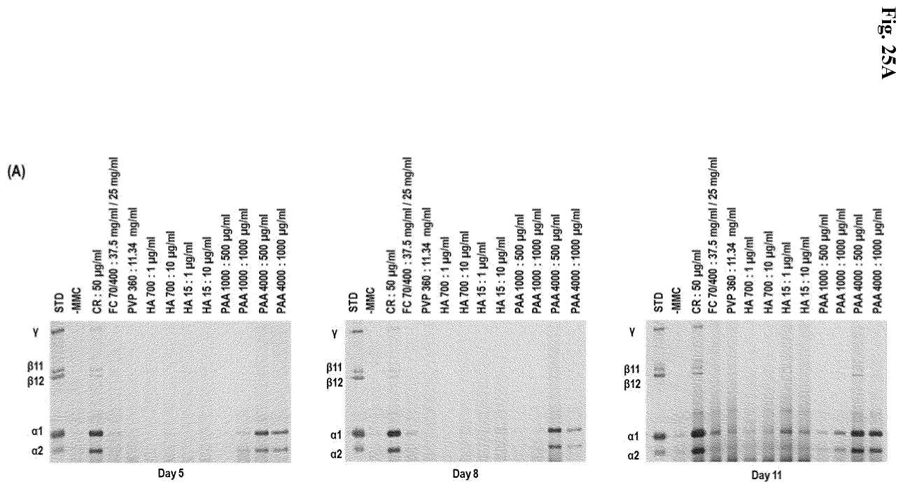

FIG. 27: Immunocytochemistry analysis of collagen type I for Donor 2 at days 5, 8 and 11 of BM-MSCs cultured without MMC (−MMC) and in the presence of crowders. Collagen type I: Green, Nuclei: Blue. Scale bars: 100 μm.

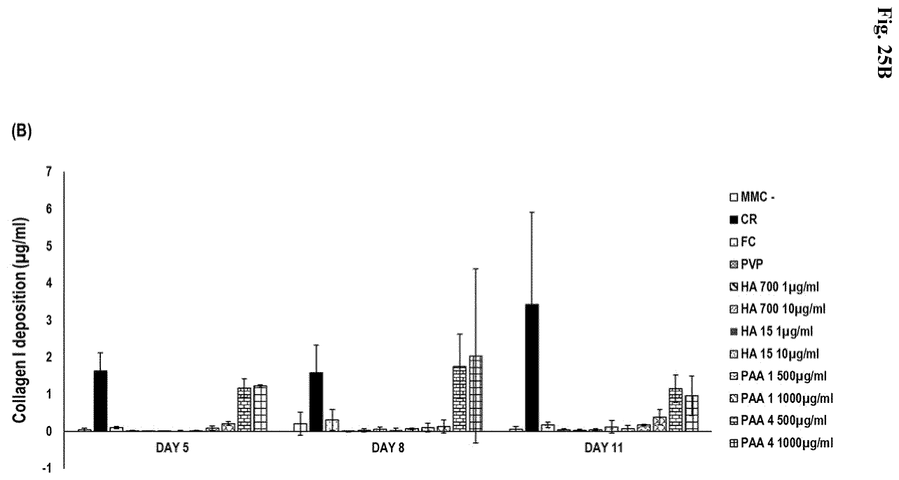

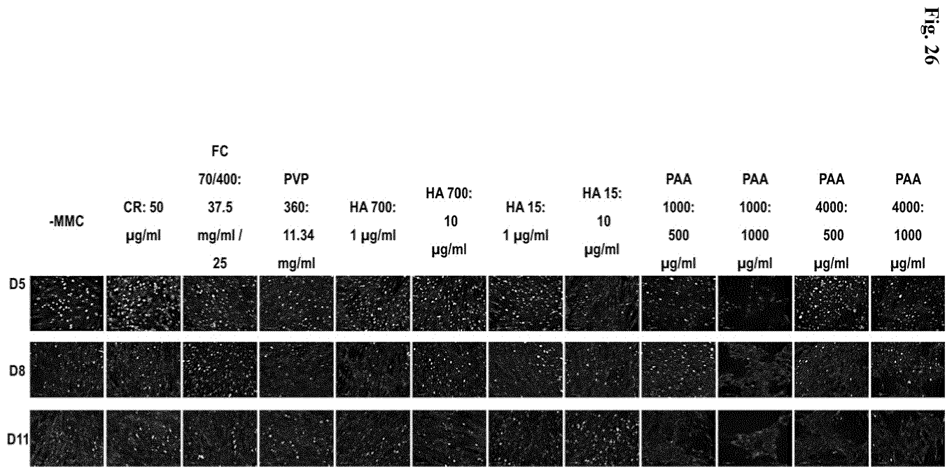

FIG. 28: Immunocytochemistry analysis of collagen type III for Donor 1 at days 5, 8 and 11 of BM-MSCs cultured without MMC (−MMC) and in the presence of crowders. Collagen type III: Green, Nuclei: Blue. Scale bars: 100 μm.

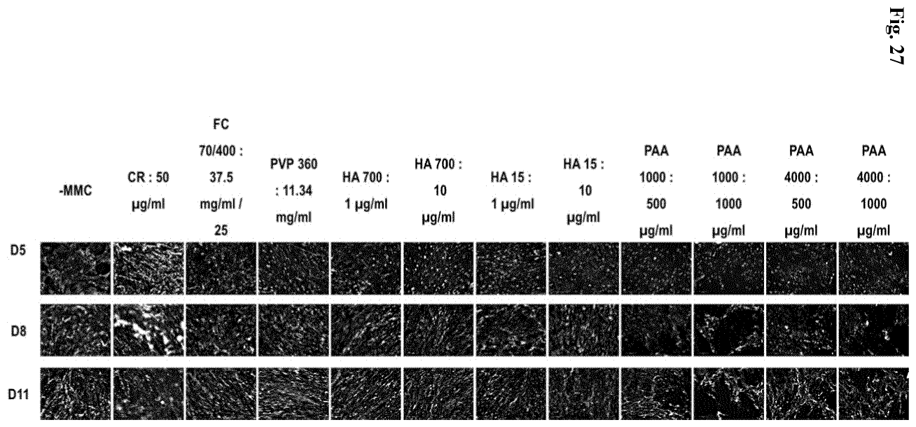

FIG. 29: Immunocytochemistry analysis of collagen type III for Donor 2 at days 5, 8 and 11 of BM-MSCs cultured without MMC (−MMC) and in the presence of crowders. Collagen type III: Green, Nuclei: Blue. Scale bars: 100 μm.

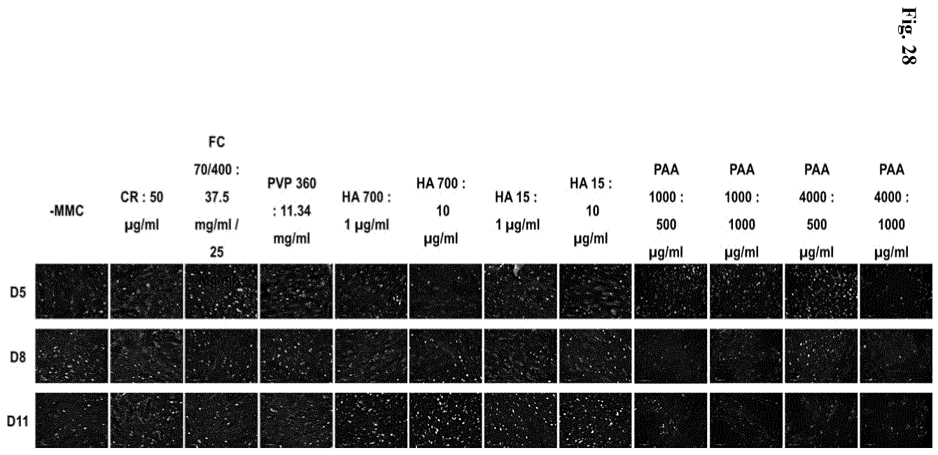

FIG. 30: Relative fluorescent intensity analysis for Donor 1 normalised to cell number for Collagen type I (A) and Collagen type III (B).

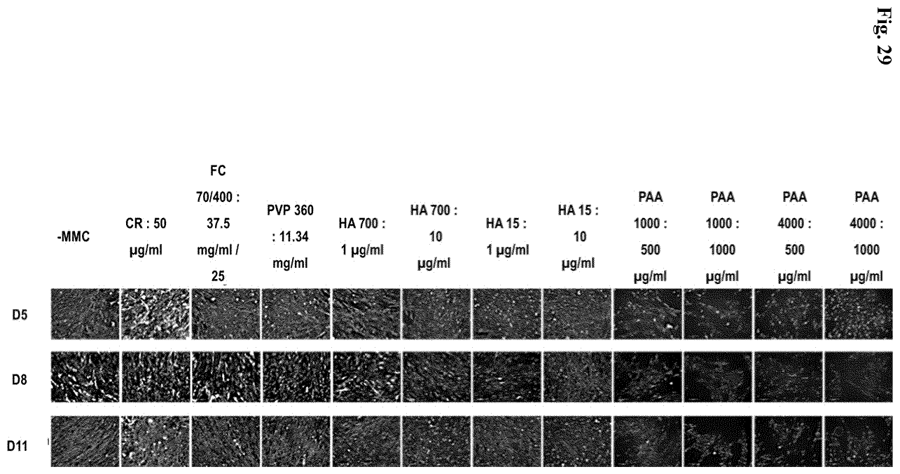

FIG. 31: Relative fluorescent intensity analysis for Donor 2 normalised to cell number for Collagen type I (A) and Collagen type III (B).

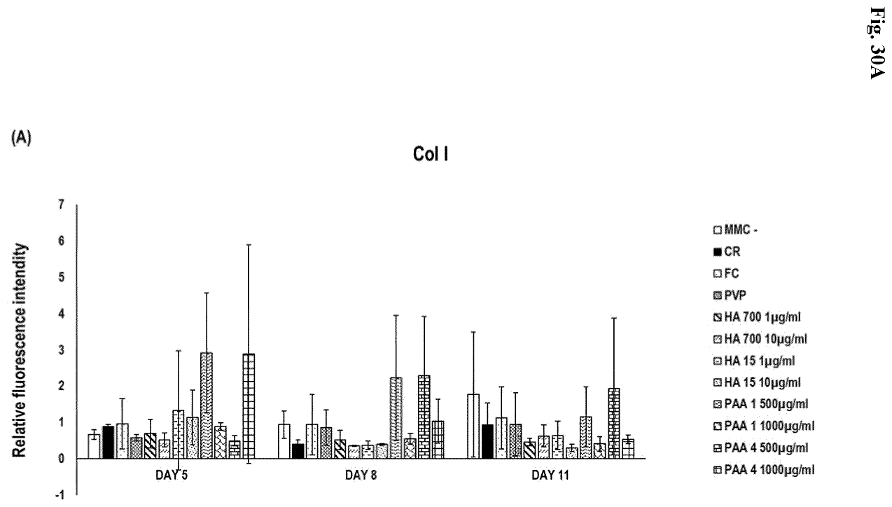

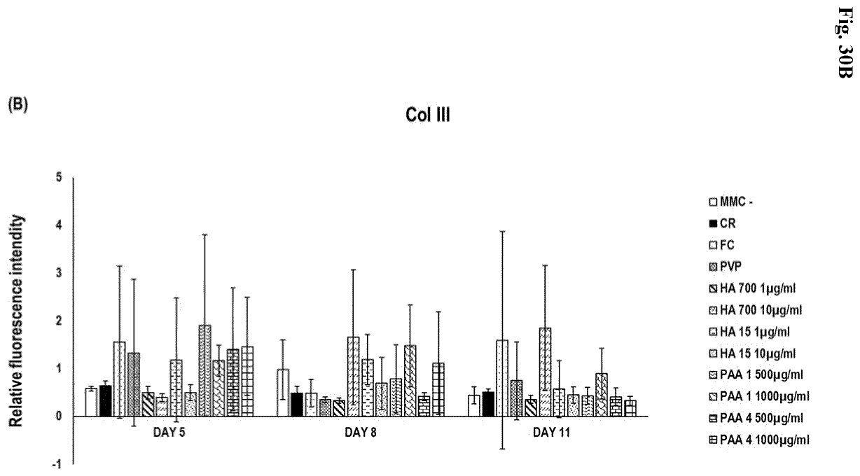

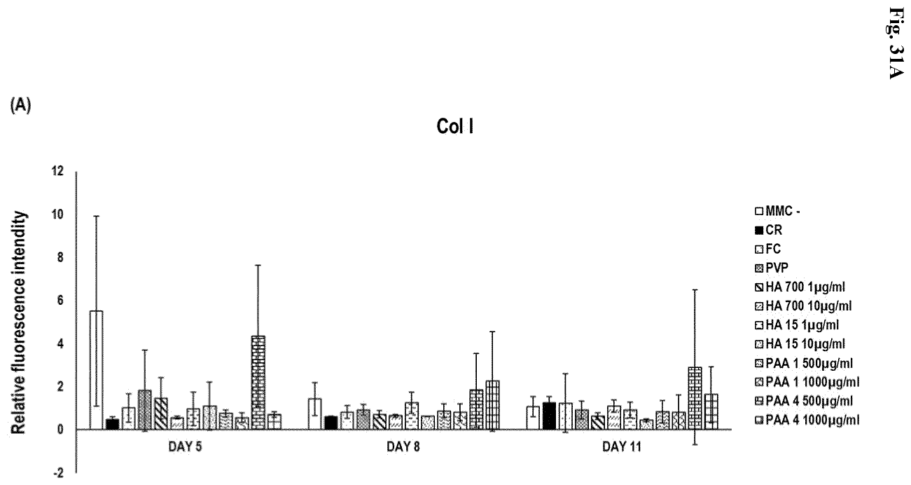

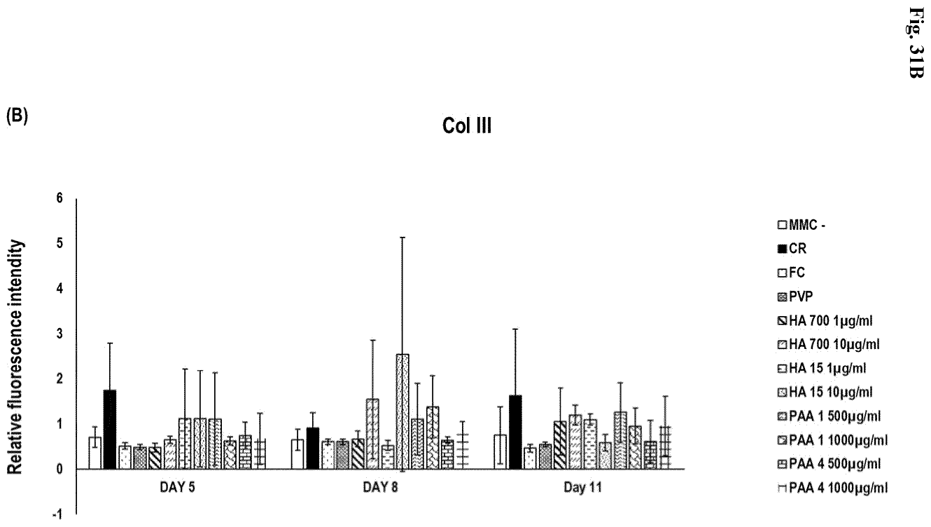

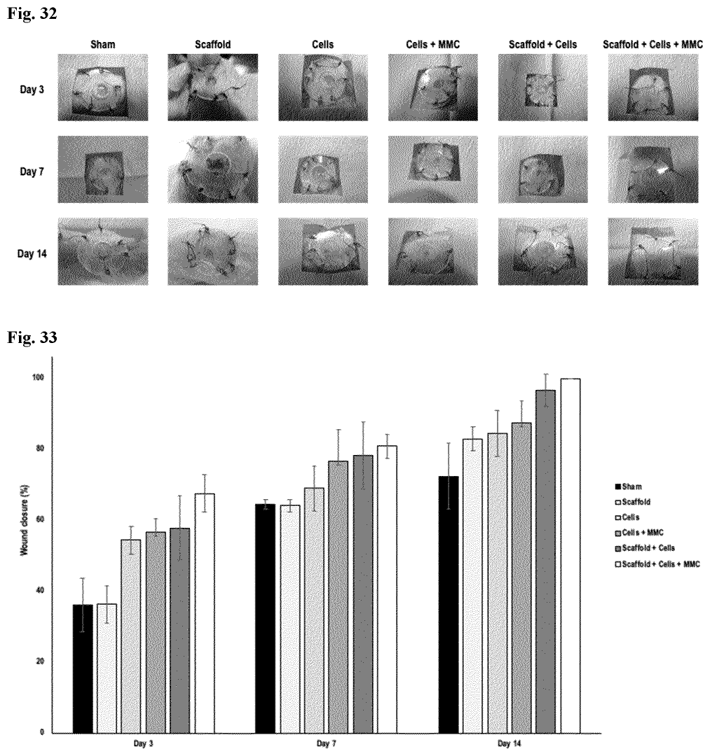

FIG. 32: Qualitative wound closure analysis showed that the Scaffold+Cells+MMC group induced the highest wound closure.

FIG. 33: Quantitative wound closure analysis revealed that the Scaffold+Cells+MMC group induced the highest wound closure.

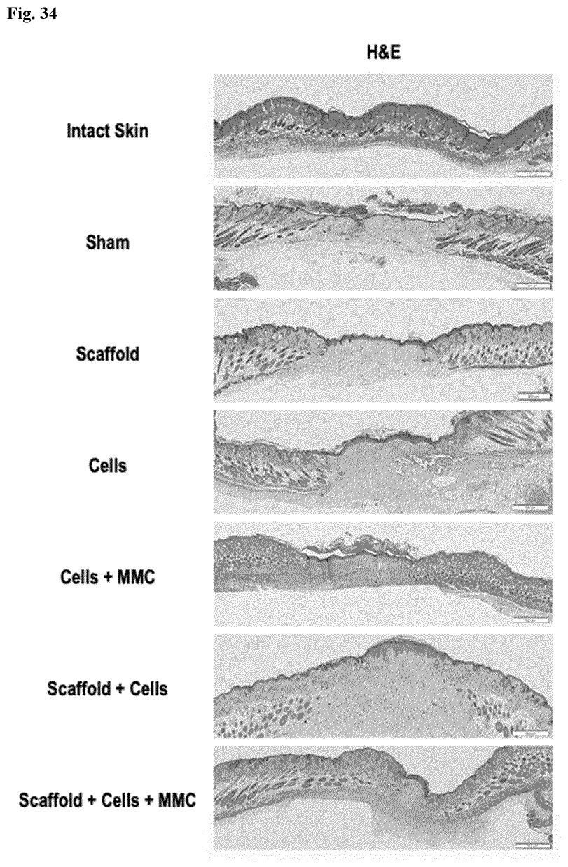

FIG. 34: H&E staining showed complete re-epithelialisation in all groups 14 days after injury.

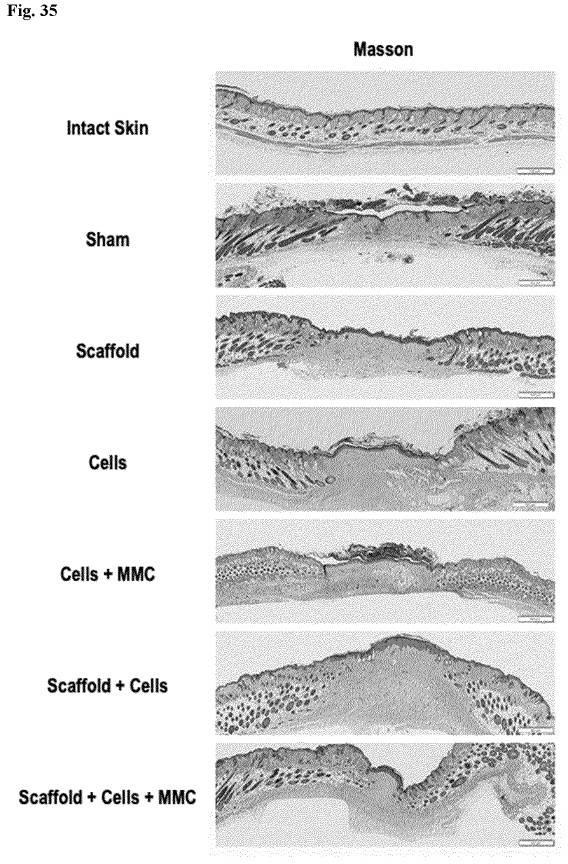

FIG. 35: Masson's trichrome staining revealed dense collagenous tissue formation in all groups with cells, but not in the Sham and Scaffold groups.

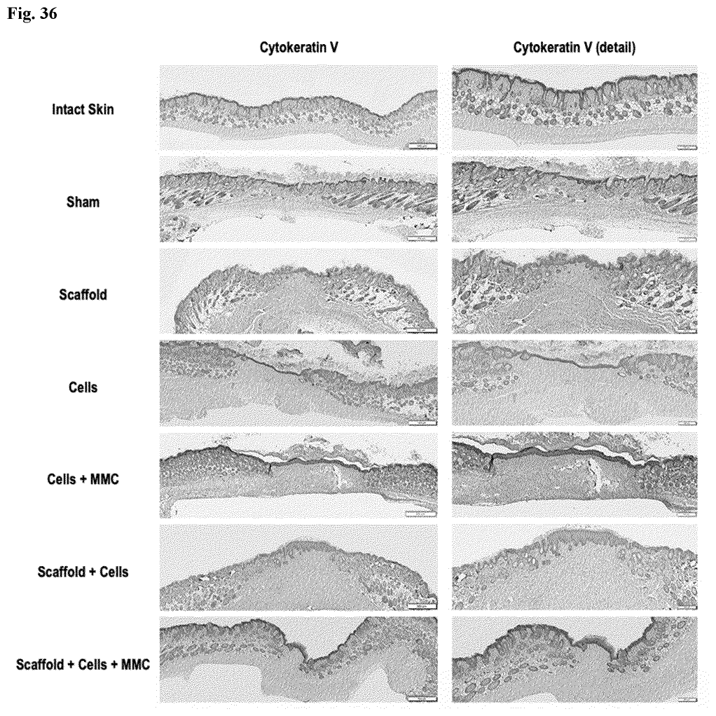

FIG. 36: Immunohistochemical analysis of cytokeratin 5 revealed that in all groups, protein expression was restricted to the epidermallayers and hair follicles. The Scaffold+Cells+MMC and Scaffold+Cells groups appeared to substantially increase the number of hair follicles, in comparison to all other groups.

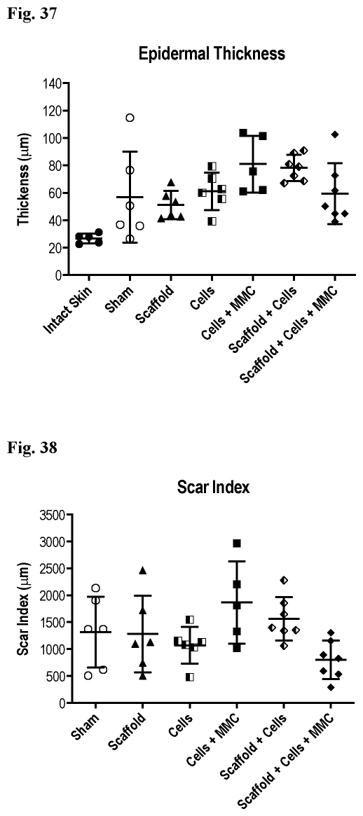

FIG. 37: Quantitative epidermal thickness analysis showed that the Cells and MMC and the Scaffold+Cells groups had a significantly (p<0.05) higher epidermal thickness compared to Intact Skin group and the Sham, Scaffold, Cells and Scaffold+Cells+MMC groups did not significantly (p>0.05) differ from the Intact Skin group.

FIG. 38: Qualitative scar index analysis revealed that the Scaffold+Cells+MMC group induced the lowest scar index.

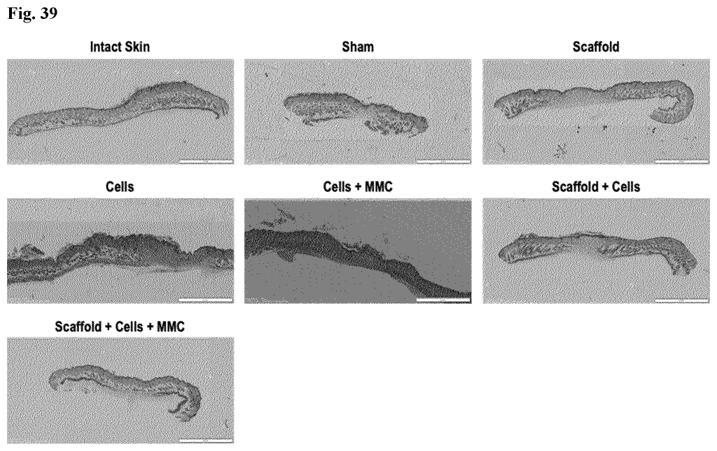

FIG. 39: Qualitative H&E analysis made apparent that in some cases, only the Scaffold+Cells+MMC group induced scarless healing.

### DETAILED DESCRIPTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”. Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### 3D Tissue Production Using Large-Polydispersed Macromolecules as Macromolecular Crowders

### Materials and Methods

### Materials

All chemicals, cell culture media and reagents were purchased from Sigma Aldrich (Ireland), unless otherwise stated. Tissue culture consumables were purchased from Sarstedt (Ireland) and NUNC (Denmark).

### Synthesis and Characterisation of Temperature-Responsive Copolymers

Poly-N-isopropylacrylamide-N-tert-butylacrylamide (pNIPAM-NTBA) copolymers were synthesised and characterised as has been described before [9, 10]. Briefly, the copolymers were prepared by free radical polymerisation using azobisisobutyronitrile as an initiator in benzene. After polymerisation at 60° C. for 24 h, the mixture was precipitated in n-hexane. The obtained copolymers were then purified by dissolving in acetone followed by precipitation in n-hexane for at least 3 times and the product was dried at 45° C. in a vacuum oven. The composition of copolymers (85 to 15 and 65 to 35 pNIPAM to NTBA) was confirmed by 1H-NMR spectroscopy. The number-average molecular weight (345,000 g/mol) and polydispersity (1.6) of the copolymers were determined by size exclusion chromatography in respect to polystyrene standards.

Size exclusion chromatography (SEC) of temperature-responsive copolymers was performed using an Ultimate 3000 Thermo Fisher Scientific chromatographic complex equipped with PLgel precolumn guard (size 7.5×50 mm, particle size 5 m, Agilent, Ireland) and PLgel MIXED-C column (size 7.5×300 mm, particle size 5 m, Agilent, Ireland) thermostated at 50° C. The elution was performed in the isocratic mode with dimethylformamide (HPLC isocratic grade, Carlo Erba, Spain) containing 0.10 M LiBr (99+%, for analysis, anhydrous, Acros Organic, Thermo Fisher Scientific, Ireland) at a flow rate of 1 ml/min. SEC traces were recorded on the refractive index detector at 40° C. The molecular weight characteristics of the polymers were calculated using Chromeleon 7.0 program (Dionex™, Thermo Fisher Scientific, Germany) based on polystyrene standards (EasiCal™, Agilent, Ireland) with Mw/Mn≤1.05. 1H NMR (500 MHz) spectra were recorded in deuterated chloroform at 25° C. on a Bruker (UK) AC-500 spectrometer calibrated relative to the residual solvent resonance.

### Fabrication of Electrospun Scaffolds

Typical protocols for electrospinning were utilised [12, 13]. Briefly, 150 mg/ml of pNIPAM and 85:15 and 65:35 pNIPAM-NTBA were dissolved in methanol (Honeywell, Ireland) and the solution was extruded at 20 μl/min through an 18 G stainless steel blunt needle (EDF Nordson, Ireland). Upon application of high voltage (20 kV) between the needle and the aluminium collector (20 cm distance), the solvent evaporated and the electrospun fibres were collected on a rotating (50 revolutions per min) mandrel. All electrospinning experiments were carried out at room temperature (22° C. to 26° C.) and 40 to 55% relative humidity.

### Electrospun Scaffold Fibre Morphology Analysis

The electrospun scaffolds were mounted onto carbon disks, gold sputter coated and imaged with a Hitachi S-4700 scanning electron microscope (Hitachi High-Technologies Europe GmbH, Germany). Fibre diameter analysis was conducted using the ImageJ software (NIH, USA).

### Electrospun Scaffold Stability and Swelling Analyses

The stability and swelling properties of the electrospun scaffolds were investigated using square samples (2 cm×2 cm). For stability analysis, each sample was submerged in phosphate buffered saline (PBS) at 37° C. and after 1 h, images were taken using a digital camera (iPhone 6, USA). For swelling analysis, each sample was weighed and then submerged in PBS at 37° C. to allow water uptake. At time intervals of 3, 6, 24, 48 and 72 h, specimens were removed from PBS and prior to weighing of the samples, the excess PBS was removed with tissue paper. The swelling ratio was calculated using the following formula: Swelling Ratio % (S)=[(Ww−Wd)/Wd]×100, where W_{w} stands for wet weight and W_{d} stands for dry weight of the samples.

### Electrospun Scaffold Contact Angle Analysis

Sessile-drop experiments were performed with a contact-angle measuring system (Acam D-2, Apex Instruments, India). During the entire test period, the samples were placed on a heated platform with moisture content level maintained at 70%. Deionised water was dropped onto the sample surface from a micro-syringe needle (volume: 10 μl, dispensing rate: 15 l/min). Droplet pictures were taken after the drop touched the sample with a periodicity of 5 see for 15 min. The contact angles were calculated by the instrument's software through analysing the shape of the drop by the tangent fitting method.

### Cell Culture

The scaffolds were cut and fixed to the bottoms of 24-well cell culture plate using silicone O-rings. The sterilisation was conducted under UV light for 2 h. Human adipose derived stem cells (hADSCs, RoosterBio, USA) were cultured in alpha-Minimum Essential Medium (α-MEM) with Gibco® GlutaMAX™ (Thermo Fisher Scientific, Ireland) supplemented with 10% foetal bovine serum (FBS) and 1% penicillin/streptomycin (P/S) at 37° C. in a humidified atmosphere of 5% CO₂. At passage 3-5, cells were seeded at 25,000 cells/cm² in 24 well plates and at 50,000 cells/cm² on the temperature-responsive electrospun scaffolds and were allowed to attach. After 24 h, the media were changed to media without/with MMC (carrageenan at 50 μg/ml). Supplementation with 100 μM of L-ascorbic acid 2-phosphate sesquimagnesium salt hydrate was used to induce collagen synthesis. Media were changed every 3 days. Samples were analysed at days 4, 7 and 10.

### Phase Contrast Microscopy Analysis

Phase contrast images were obtained using an inverted microscope (Leica Microsystems, Germany) at each timepoint. Images were processed using ImageJ software (NIH, USA).

### Cell Morphology Analysis

At each timepoint, cells were fixed with 4% paraformaldehyde, permeabilised with 0.2% Triton X-100 and stained with FITC-labelled phalloidin (Thermo Fisher Scientific, UK) for the cytoskeleton and Hoechst for the nucleus. Samples were imaged in an inverted fluorescence microscope (Olympus IX81, Olympus Corporation, Japan).

### Cell Viability Analysis

At each timepoint, cells were washed with PBS and a solution of Calcein AM (4 mM, Thermo Fisher Scientific, UK) and ethidium homodimer I (2 mM, Thermo Fisher Scientific, UK) was added. Cells were incubated at 37° C. and 5% CO₂ for 30 min after which, fluorescence images were obtained with an Olympus IX-81 inverted fluorescence microscope (Olympus Corporation, Japan).

### Cell Metabolic Activity Analysis

The alamarBlue® assay (Invitrogen, USA) was used to quantify cell metabolic activity as per manufacturer's protocol. Briefly, at each timepoint, cells were washed with PBS and alamarBlue® solution (10% alamarBlue® in PBS) was added. After 4 h of incubation at 37° C., absorbance was measured at excitation wavelength of 550 nm and emission wavelength of 595 nm using a Varioskan Flash spectral scanning multimode reader (Thermo Fisher Scientific, UK). Cell metabolic activity was expressed as % reduction of the alamarBlue® and normalised to non-MMC control group.

### DNA Quantification Analysis

DNA quantification was assessed using the Quant-iT™ PicoGreen® dSDNA assay kit (Invitrogen, Ireland) according to the manufacturer's protocol. Briefly, DNA was extracted using a papain extraction reagent for 3 h at 65° C. 28.7 μl were then transferred into 96-well plates. A standard curve was generated using 0, 100, 200, 375, 500, 1,000, 2000 and 4000 ng/ml DNA concentrations. 71.3 μl of a 1:200 dilution of Quant-iT™ PicoGreen® reagent were added to each sample and the plate was read using a using a Varioskan Flash spectral scanning multimode reader (Thermo Fisher Scientific, UK) with an excitation wavelength of 480 nm and an emission wavelength of 525 nm.

### Cell Sheet Detachment Analysis

At each timepoint, electrospun scaffolds with cells were rinsed with pre-warmed PBS. To induce detachment cold PBS (4° C.) was added and samples were left on a digitally controlled chilling/heating dry bath (Torrey Pines Scientific, USA) set to 4° C. Additional washes with cold PBS were repeated in order to remove any excess of polymer.

### Time Lapse Microscopy Analysis

A Linkam THMS600 Heating and Freezing microscope stage (Linkam Scientific Instruments, UK) was attached to a BX51 Olympus microscope (Olympus Corporation, Japan). Cell detachment was conducted as described above. Images were taken every 5 sec until full dissolution of the electrospun scaffolds.

### Sodium Dodecyl Sulphate-Polyacrylamide Gel Electrophoresis (SDS-PAGE) Analysis

SDS-PAGE was conducted as has been described previously [14]. Briefly, at each timepoint cell layers were digested with pepsin from porcine gastric mucosa at 0.1 mg/ml in 0.5 M acetic acid for 2 h at 37° C. with continuous shaking and subsequent neutralisation with 1 N NaOH. The samples for SDS-PAGE were prepared using appropriate dilution with distilled water and 5× sample buffer. 10 μl per sample solution per well were loaded on the gel (5% running gel/3% stacking gel) after 5 min heating at 95° C. Electrophoresis was performed with a Mini-PROTEAN Tetra Electrophoresis System (Bio-Rad, Ireland) by applying a potential difference of 50 mV for the initial 30 min and 120 mV for the remaining time (approximately 1 h). The gels were stained using a silver stain kit (SilverQuest™, Invitrogen, Ireland) according to the manufacturer's protocol. Images of the gels were taken after brief washing with water. To quantify the cell-produced collagen type I deposition, the relative densities of collagen α1(I) and α2(I) chains were evaluated with ImageJ software (NIH, USA) and compared to the α1(I) and α2(I) chain bands densities of standard collagen type I (Symatese Biomateriaux, France).

### Histological Analysis

At each timepoint, the cell sheets were detached from the electrospun scaffolds, fixed in 4% paraformaldehyde for 24 h, washed with PBS, infiltrated with 15% sucrose in PBS for 12 h and in 30% sucrose in PBS overnight and embedded in Tissue Freezing Medium® (Leica Biosystems, Ireland). Subsequently, transverse cryosections of 5 m in thickness were obtained using the CM1850 Cryostat (Leica Biosystems, Ireland). The samples were then stained with haematoxylin-eosin, Masson-Goldner's trichrome stain (Carl Roth, Germany), Herovici's polychrome stain and Picrosirius red stain according to the manufacturer's guidelines and mounted using DPX mountant. Images were captured with an Olympus IX-81 inverted microscope (Olympus Corporation, Tokyo, Japan). Cell sheet thicknesses were measured using the digitalised images and ImageJ software (NIH, USA). The same staining protocols were applied for hADSCs grown in 2D culture on glass coverslips as control.

### Immunocytochemistry Analysis

At each timepoint, cells were briefly washed with PBS and fixed with 4% paraformaldehyde for 20 min at room temperature. Cells were washed again and non-specific binding sites were blocked with 3% bovine serum albumin (BSA) in PBS for 30 min. The cells were incubated overnight at 4° C. with one of the following primary antibodies: mouse anti-collagen type I, rabbit anti-collagen type III, rabbit anti-collagen type V and rabbit anti-fibronectin. After 3 washes in PBS, cells were incubated for 30 min at room temperature with the secondary antibody AlexaFluor® 488 goat anti-rabbit (Invitrogen, USA). The cell nuclei were stained with Hoechst. Images were taken with an Olympus IX-81 inverted fluorescence microscope (Olympus Corporation, Japan). The same staining protocol was applied on cryosections, which were mounted with Fluoromount™ Aqueous Mounting Medium and left for 2 h at room temperature to dry before imaging.

### Atomic Force Microscopy (AFM) Analysis

AFM analysis was performed as per previously published protocol [15]. Briefly, freshly cut 5 μm thick sections were attached directly onto 13 mm diameter glass coverslips. Prior to imaging, the sections were thawed, air-dried, washed with water to remove the support medium and air-dried again. Samples were imaged by intermittent contact mode in air using a Dimension 3100 AFM (Veeco, UK) with a Nanoscope IIIa controller and a 12 μm×12 μm×3.2 m (X, Y, Z dimension) E scanner. Height, amplitude and phase images at scan sizes of 1 μm or 5 μm were captured at an initial scan rate of 1.97 Hz and integral and proportional gain settings of 0.3 and 0.5, respectively.

### Trilineage Differentiation Analysis

For all differentiation experiments cells at passage 5 were seeded at a density of 50,000 cells/cm². Osteogenic, adipogenic and chondrogenic assays were performed without (−) or with (+) MMC in the differentiation media. Osteogenic, adipogenic and chondrogenic differentiations were initiated 24 h after seeding and cells were differentiated for 21 days. As control, cells were grown on tissue culture plastic (TCP) for osteogenic and adipogenic differentiation and as pellets for chondrogenic differentiation. Osteogenesis was induced using media composed of α-MEM with Gibco® GlutaMAX™ (Thermo Fisher Scientific, Ireland) supplemented with 10% FBS, 1% P/S, 50 μM L-ascorbic acid 2-phosphate sesquimagnesium salt hydrate, 10 mM β-glycerophosphate, 100 μM dexamethasone, with or without MMC. Adipogenesis was induced through cycles by 7 days of induction media composed of Dulbecco's modified Eagle medium high glucose (DMEM-HG), supplemented with 10% FBS, 1% P/S, 1 μM rosiglitazone, 1 μM dexamethasone, 0.5 mM 3-isobutyl-1-methylxanthine, 10 μg/ml insulin, with or without MMC and subsequently with maintenance media composed of DMEM-HG, supplemented with 10% FBS, 1% P/S and 10 μg/mL insulin, with or without MMC.

Chondrogenesis was induced using media composed of DMEM-HG, supplemented with 10 ng/ml transforming growth factor 03 (PromoCell GmbH, Germany), 100 nM dexamethasone, 10% insulin-transferrin-selenium, 40 μg/ml L-proline, 100 μM L-ascorbic acid 2-phosphate sesquimagnesium salt hydrate, with or without MMC.

### Alizarin Red Staining Analysis

Samples were fixed with ice-cold methanol for 20 min, stained with 2% alizarin red in deionised water for 10 min and washed with water. Images were acquired using an inverted microscope (Leica Microsystems, Germany). Semi-quantitative analysis of alizarin red staining was performed by dissolving the bound stain with 10% acetic acid. Samples were collected using a cell scraper and heated to 85° C. for 10 min. Subsequently 10% solution of ammonium hydroxide was used to achieve a pH of 4.5. Finally, the absorbance was read at 405 nm using a micro-plate reader (Varioskan Flash, Thermo Fisher Scientific, Ireland).

### Oil Red O Staining Analysis

Samples were fixed for 20 min with 4% paraformaldehyde, stained for 10 min with oil red O solution (oil red O 0.5% in isopropanol, diluted 3:2 in distilled water) at room temperature and images were acquired using an inverted microscope (Leica Microsystems, Germany). For quantification of oil red O staining, the dye was extracted pipetting 100% isopropanol over the surface of the wells. Then, the solution was centrifuged at 500×g for 2 min to remove debris and the absorbance was measured at 520 nm using a Varioskan Flash spectral scanning multimode reader (Thermo Fisher Scientific, Ireland).

### Sulphated Glycosaminoglycan Analysis

Cell layers and culture media were digested for 3 h at 60° C. with 0.1% crystallised papain in 0.2 M sodium phosphate buffer at pH 6.4, containing sodium acetate, ethylenediaminetetraacetic acid (EDTA), disodium salt and cysteine-HCl. For sulphated glycosaminoglycan (sGAG) quantification, the Blyscan™ Glycosaminoglycan Assay (Biocolor, UK) was used, as per manufacturer's protocol.

### Growth Factors and Matrix Metalloproteinases Analysis

The expression of growth factors from conditioned media and cell layers of hADSCs cultured both on TCP and 85:15 pNIPAM-NTBA without and with MMC and the expression of matrix metalloproteinases (MMPs) from conditioned media and cell layers of hADSCs cultured on 85:15 pNIPAM-NTBA without and with MMC were assessed using antibody arrays (Abcam, UK), following the manufacturer's protocol. Briefly, hADSCs were cultured for 10 days without and with MMC. For the protein extraction from the cell layers, radioimmunoprecipitation assay buffer with proteinase and phosphatase inhibitor cocktail was added to the cell layers and left to incubate at 4° C. for 30 min, after which cell layers were scratching collected, centrifuged and frozen at −80° C. 6 replicates were pooled prior to total protein quantification, which was performed using the Pierce™ BCA Protein Assay Kit (Thermo Fisher Scientific, UK) following the manufacturer's protocol. Protein concentration was determined using a BSA standard curve. For the conditioned media, at day 10, the culture media was removed and replaced with fresh media containing 0.2% FBS, which was subsequently collected after 3 days. 6 replicates were pooled prior to analysis. The antibody membranes were incubated overnight with 1 ml of conditioned media or 250 μg of proteins. The array membranes were developed using an enhanced chemiluminescence method according to the manufacturer's protocol. The relative expression of the growth factors and the MMPs was determined by measuring the pixel intensity of each chemiluminescence image.

### Scratch and Migration Assays Analysis

To analyse the effect of MMC on cell migration, a scratch wound healing assay [16] and a migration assay were performed. For the scratch assay, hADSCs were cultured for 10 days with and without MMC. Before starting the assay, cells were serum starve for 16 h with media containing 0.2% FBS. Two perpendicular scratches were created with a P10 pipette tip. To remove detached cells and proteins after wound creation, cells were rinsed once with PBS and new media containing 0.2% FBS was added. Images were obtained at 0, 24, 48 and 72 h. The migration assay was performed using a 2 well silicone insert with a defined cell-free gap (IBIDI, Germany). Values were reported as percent of wound closure and calculated as follows: [(area of original wound/gap−area of actual wound/gap)/area of original wound/gap]×100.

### Mouse Excisional Wound Splinting Model

All animal experiments and procedures were conducted in accordance to Irish laws on animal experimentation and were approved by the Animal Care and Research Ethics Committee of NUI Galway and the Irish Health Products Regulatory Authority (License Number: AE 19125/P051). A total of 18 female athymic nude mice (6-7 weeks old), purchased from Charles River (UK), were included in the study. The mice were kept in individually ventilated cages with controlled temperature and humidity and a 12 h light-dark cycle. All animals were housed in groups prior to surgery and were provided access to food and water ad libitum. Postoperatively, the animals were housed individually to avoid chewing of wounds and bandages. After one-week acclimatisation period, animals were randomly divided into the following three groups: non-treated control (n=6), cell sheets without MMC (−MMC, n=6) and cell sheets with MMC (+MMC, n=6). Every animal received perioperative analgesia with a subcutaneous injection of buprenorphine (0.05 mg/kg, Bupaq®, Chanelle Pharma Group, Ireland) 1 hour prior to surgical anaesthesia. Anaesthesia was induced and maintained with isoflurane (Iso-Vet®, Chanelle Pharma Group, Ireland). A splinted wound healing model was utilised. Briefly, the surgical field at the back of each mouse was cleaned with iodine scrub and 70% ethanol solution. The skin was folded and two circular full thickness (epidermis, dermis, subcutaneous tissue and panniculus carnosus muscle) wounds of 5 mm diameter were created with a single puncture using a punch biopsy (KAI Medical, Italy). A silicone splint with internal and external diameter of 6 mm and 12 mm, respectively, and 0.5 mm thickness (Grace Bio-Labs, USA) was sutured around every wound to prevent contraction and promote healing by epithelisation. An identical treatment was applied to both wounds of each mouse. Animals received twice per day a subcutaneous injection of buprenorphine (0.05 mg/kg) for post-operative analgesia and once per day a subcutaneous injection of enrofloxacin (5 mg/kg, Baytril®, Bayer, Germany) as antibiotic treatment for 3 days.

### Cell Labelling and In Vivo Cell Tracking Analysis

Before implantation, cell sheets were incubated with 10 μM Vybrant™ DiD fluorescent dye (Thermo Fisher Scientific, Ireland) overnight at 37° C. and then washed 3 times for 5 min each with sterile PBS. Before imaging, animals were anesthetised with isoflurane. The wavelengths of absorption were set up at 644 nm excitation and 665 nm emission. Digital pictures of animals were taken immediately post-surgery and at days 3, 7, 10 and 14 with the Spectrum In Vivo Imaging System IVIS® Lumina III (PerkinElmer, UK). Average radiant efficiency was calculated with Living Image Software® (IVIS Imaging Systems, UK).

### Wound Healing Ratio Analysis

Wound closure rate was determined by taking digital pictures of the wounds with an iPad Pro (Apple, USA) immediately post-surgery and at days 3, 7, 10 and 14. The planimetric area of the open wounds was measured using the software WoundWise IQ (Med-Compliance IQ, USA). Values were reported as % of wound closure and calculated as follows: [(area of original wound−area of actual wound)/area of original wound]×100.

### Histological Analysis

After euthanasia, skin tissue samples were harvested and fixed in 4% paraformaldehyde for paraffin embedding. 5 m in thickness cross-sections were prepared from all paraffin blocks. The sections were deparaffinised with 2 immersions in xylene and re-hydrated with descending concentrations of ethanol (100%, 90%, 70%, 0% in distilled water). Sections were stained using haematoxylin-eosin, Masson-Goldner's trichrome stain (Carl Roth, Germany), Herovici's polychrome stain and Picrosirius red according to the manufacturer's protocols and mounted using DPX mountant. Slides were scanned and images were captured using an Olympus VS120 virtual slide microscope and OlyVIA software and an Olympus BX51 microscope for polarised microscopy analysis (all Olympus Corporation, Japan).

### Immunohistochemistry Analysis

Paraffin sections were dewaxed and re-hydrated as described above. Endogenous peroxidases were blocked by incubating the samples in 3% hydrogen peroxide in 100% methanol for 20 min. Antigen retrieval was carried out in a pressure cooker in 0.01 M Tris-EDTA (pH 9.0). The slides were then incubated for 30 min at room temperature in antigen blocking solution (5% normal goat serum and 0.1% Triton X-100 in PBS). Slides were incubated overnight at 4° C. with the following primary antibodies: rabbit anti-cytokeratin 5, rabbit anti-CD 31 and mouse anti-human nuclear antigen. Secondary antibodies, biotinylated swine anti-rabbit and biotinylated rabbit anti-mouse (Dako, USA), were added and the slides were further incubated for 1 h at room temperature. For the detection, ABC horseradish peroxidase labelled Vectastain Elite ABC reagent (Vector, UK) was used. Binding sites of primary antibodies were visualised using diaminobenzidine (Dako, UK) as chromogen and all sections were counterstained with haematoxylin. Negative controls were prepared for each stain by omitting primary antibodies during incubation, which resulted in no staining. Human skin tissue sections were used as positive controls for anti-human nuclear antigen antibody. Images were captured using an Olympus VS120 digital scanner and OlyVIA software (both Olympus Corporation, Japan).

### Epidermal Thickness Analysis

The thickness of the neo-formed epidermis was evaluated with ImageJ (NIH, USA) using Masson-Goldner's trichrome stained histological sections. Beginning from the centre of the wound, 3 non-consecutive sections (100 μm distance from each another) per group, were analysed by randomly selecting 3 high-power fields and performing 5 measurements of the epidermal thickness per field.

### Scar Size Analysis

Scar size analysis was performed as per established protocols [16]. Briefly, scar area was evaluated using Masson-Goldner's trichrome stained histological sections. Beginning from the centre of the wound, 3 non-consecutive sections per group, with a distance of 100 μm, were analysed by randomly selecting 3 high-power fields and performing 5 measurements of the scar size per field. Scar tissue was outlined using the freeform outline tool in ImageJ (NIH, USA) to produce a pixel-based area measurement, which then converted to m². Scar area measurements were performed extended to the panniculus carnosus. A positive and predictive relationship was established between dermal thickness and scar area. Scar size was determined by the scar index, which was calculated as follow: scar index (m)=scar area (m²)/average dermal thickness (m). Dermal thickness measurements were obtained using ImageJ (NIH, USA) by drawing a line normal to the average orientation of the epidermal-dermal and dermal-subcutaneous tissue demarcations. 4 dermal thickness measurements were taken per sample, two adjacent to the wound site at 50 μm on either side, and two at a farther distance of 700 μm on either side of the wound.

### Statistical Analysis

Data are expressed as mean±standard deviation. All experiments were conducted at least in triplicates. Statistical analysis was performed using MINITAB® version 19 (Minitab Inc., USA). One-way analysis of variance (ANOVA) was used for multiple comparisons and Tukey's post hoc test was used for pairwise comparisons after confirming the samples followed a normal distribution (Anderson-Darling test) and had equal variances (Bartlett's and Levene's test for homogeneity of variances). When either or both assumptions were violated, non-parametric analysis was conducted using Kruskall-Wallis test for multiple comparisons and Mann-Whitney test for pairwise comparisons. Statistical significance was accepted at p<0.05.

### Results

### Morphology, Stability, Swelling, Contact Angle and Dissolution Analyses of Temperature-Responsive Electrospun Scaffolds

Scanning electron microscopy analysis (pNIPAM, 85:15 pNIPAM-NTBA, 65:35 pNIPAM-NTBA) revealed that all electrospun scaffolds were composed of uniform (bead-free) randomly oriented fibres. Fibre diameter distribution analysis showed that the pNIPAM scaffolds were comprised of fibres with diameter range from 300 nm to 600 nm and the 85:15 pNIPAM-NTBA and 65:35 pNIPAM-NTBA scaffolds were comprised of fibres with diameter range from 1,000 nm to 2,000 nm.

Stability analysis revealed that the pNIPAM (LCST 32° C.) electrospun scaffolds were completely soluble, whilst the 85:15 pNIPAM-NTBA (LCST 25° C.) and the 65:35 pNIPAM-NTBA (LCST 16° C.) electrospun scaffolds were stable in PBS at 37° C.

Swelling ratio analysis showed that the 85:15 pNIPAM-NTBA electrospun scaffolds had swelling ratio of 5% up to 24 h and then increased to 35% until the end of the experiment (72 h) and the swelling ratio of the 65:35 pNIPAM-NTBA electrospun scaffolds did not exceed the 4% for the duration of the experiment (72 h).

Static contact angle measurements against deionised water made apparent that the 85:15 pNIPAM-NTBA electrospun scaffolds had average contact angle of 74°±4° and the 65:35 pNIPAM-NTBA electrospun scaffolds had average contact angle of 81°±2°.

Time-lapse microscopy revealed that the 85:15 pNIPAM-NTBA electrospun scaffolds were dissolved in fast and uniform manner and the 65:35 pNIPAM-NTBA electrospun scaffolds were dissolved slowly in a layer-by-layer fashion.

### Cell Attachment, Spreading, Viability, DNA Concentration and Metabolic Activity Analyses and Cell Sheet Detachment Analysis

Qualitative rhodamine-phalloidin staining showed that at all timepoints and without and with MMC the 85:15 pNIPAM-NTBA and the 65:35 pNIPAM-NTBA electrospun scaffolds supported hADSC attachment and spreading in similar manner to the control TCP.

Qualitative and quantitative hADSC viability analyses revealed no apparent differences (p>0.05) at any timepoint and without and with MMC between the control TCP and the 85:15 pNIPAM-NTBA and the 65:35 pNIPAM-NTBA electrospun scaffolds.

Cell sheet detachment from the 85:15 pNIPAM-NTBA electrospun scaffolds ranged from 5 min to 10 min and from the 65:35 pNIPAM-NTBA electrospun scaffolds ranged from 3 to 4 h (data not shown); for this reason, all subsequent experiments were conducted only with the 85:15 pNIPAM-NTBA electrospun scaffolds.

Qualitative image analysis made apparent that intact cell sheets without and with MMC were obtained from the 85:15 pNIPAM-NTBA electrospun scaffolds at all timepoints and qualitative phase contrast microscopy analysis showed minimal cell sheet shrinkage after complete detachment.

hADSC DNA concentration on TCP was not significantly (p>0.05) affected as a function of time in culture and absence or presence of MMC; on 85:15 pNIPAM-NTBA electrospun scaffolds was significantly (p<0.05) increased as a function of time in culture, but not (p>0.05) as a function of MMC. hADSC metabolic activity was not significantly (p>0.05) affected as a function of time in culture, absence or presence of MMC and culture substrate (TCP or 85:15 pNIPAM-NTBA electrospun scaffolds).

### SDS-PAGE, Histology, Immunocytochemistry and AFM Analyses

SDS-PAGE and corresponding densitometric analyses of hADSCs on TCP and on 85:15 pNIPAM-NTBA electrospun scaffolds revealed that MMC significantly (p<0.01) increased collagen type I deposition at all timepoints, which was matured as a function of time in culture, as evidenced by the presence of β- and γ-bands.

Histological analysis (FIG. 1) of hADSCs grown on TCP and on 85:15 pNIPAM-NTBA electrospun scaffolds revealed abundant ECM deposition in the presence of MMC. In particular, in the case of cell sheets derived from culturing hADSCs on 85:15 pNIPAM-NTBA electrospun scaffolds, histological analysis (FIG. 1) using haematoxylin-eosin showed that cells assembled into multiple layers, across all timepoints both without and with MMC, with the MMC groups leading to thicker tissue-like assemblies; Masson-Goldner's trichrome verified the presence of a collagen-rich ECM in the MMC groups at all timepoints; Herovici's polychrome and Picrosirius red indicated that the use of MMC resulted in cell sheets with densely packed mature collagen fibres; and polarised microscopy of Picrosirius red stained sections revealed a higher organisational order in the presence of MMC. Quantification of cell sheet thickness in the absence and presence of MMC revealed that MMC resulted in 2.7-fold at day 4, 3.4-fold at day 7 and 2.4-fold at day 10 increase in thickness in comparison to the non-MMC counterparts.

Immunocytochemistry (FIG. 2) and complementary relative fluorescence intensity analyses made apparent that, in general, at all timepoints MMC significantly (p<0.05) increased collagen type I, collagen type III, collagen type V and fibronectin deposition in hADSC cultures on 85:15 pNIPAM-NTBA electrospun scaffolds (apart from collagen type III at day 4) and on TCP (apart from collagen type III at day 7 and fibronectin at all timepoints). Further immunocytochemistry analysis of longitudinal sections of the cell sheets revealed a homogenous distribution of the collagen type I, collagen type III, collagen type V and fibronectin.

AFM analysis of hADSCs on 85:15 pNIPAM-NTBA electrospun scaffolds further corroborated the abundant deposited ECM when MMC was used and indicated physiological collagen assembly by the ample presence of quarter staggered (D-banded) fibrils.

### Trilineage Differentiation Analysis

Alizarin red staining (FIG. 3A) and corresponding absorbance quantification (FIG. 3B) revealed that on TCP the highest (p<0.05) level of osteogenesis was obtained when hADSCs were cultured with differentiation media containing MMC and on 85:15 pNIPAM-NTBA electrospun scaffolds osteogenesis was successfully obtained when hADSCs were cultured with osteogenic induction media both without and with MMC as evidenced by the significantly (p<0.05) increase in calcium nodules deposition in comparison to non-differentiated control.

Oil red O staining (FIG. 3C) and corresponding absorbance quantification (FIG. 3D) made apparent that on TCP the highest (p<0.05) level of adipogenesis was obtained when hADSCs were cultured without MMC during differentiation and on 85:15 pNIPAM-NTBA electrospun scaffolds the adipogenesis was successfully obtained when hADSCs were cultured with adipogenic induction media both without and with MMC as evidenced by the significantly (p<0.05) increase in lipid droplets accumulation in comparison to non-differentiated control.

Alcian blue staining (FIG. 3E) and GAG quantification (FIG. 3F) demonstrated that on TCP and on 85:15 pNIPAM-NTBA electrospun scaffolds the highest (p<0.05) level of chondrogenesis was obtained when hADSCs were cultured with MMC during differentiation.

### Growth Factors and Matrix Metalloproteinases Analyses

Growth factor antibody array of hADSCs cultured on TCP and complementary overall and ratio between soluble and matrix-bound growth factors quantification analyses revealed that MMC increased growth factor content in the cell layer.

Growth factor and MMP antibody array of hADSCs cultured on 85:15 pNIPAM-NTBA electrospun scaffolds and complementary overall (for growth factors: FIG. 4A, for MMPs: FIG. 4B) and ratio between soluble and matrix-bound growth factors (FIG. 4C) and ratio between soluble and matrix-bound MMPs (FIG. 4D) quantification analyses revealed that MMC increased growth factor content in the conditioned media and increased MMP content in the cell layers.

### Scratch and Migration Assay Analyses

Scratch wound healing assay and corresponding quantification analysis in hADSC cultures revealed that MMC induced significantly (p<0.05) faster gap closure within 24 h, whilst in the absence of MMC 48 h were needed. Migration assay and corresponding quantification analysis in hADSC cultures made apparent that MMC induced significantly (p<0.05) slower gap closure after 48 h and 72 h than in the absence of MMC.

### Transplantation Analysis

In vivo cell tracking (FIG. 5A) and complementary average radiance efficiency (FIG. 5B) analyses revealed that the without and the with MMC groups retained the cells at the implantation site with no apparent differences (p>0.05) between the groups. Qualitative (FIG. 5C) and quantitative (FIG. 5D) wound closure analysis revealed that at day 3, the MMC group had the highest % of wound closure, albeit it was not statistically significant (p>0.05); at day 7 and day 10, the MMC group had the highest (p<0.001) % of wound closure; and at day 14, all conditions (p>0.05) reached total wound closure.

Haematoxylin-eosin staining (FIG. 6A) showed complete re-epithelisation in all groups after 14 days. Masson's trichrome staining (FIG. 6B) revealed dense collagenous tissue formation in the cell sheet groups (without and with MMC), but not in the sham group. Herovici's polychrome staining (FIG. 6C) showed that the cell sheet groups, in particular the MMC group, induced a neotissue composed primarily of mature collagen, whilst the sham group formed neodermis primarily composed of immature collagen. Picrosirius red staining (FIG. 6D) and complementary polarised microscopy (FIG. 6E) analyses further corroborated that the cell sheet groups, especially the MMC group, had an ECM mainly composed of densely packed mature collagen fibres, whilst the sham group was composed of a loosely packed immature collagen network.

Immunohistochemical analysis of cytokeratin 5 (FIG. 6F) revealed that in the without and with MMC groups, its expression was evidenced in both epidermal and dermal layers, whilst in the sham group, its expression was restricted only to the epidermal layer and the MMC group appeared to substantially increase the number of hair follicles, in comparison to the without MMC and sham groups. The MMC group, as opposed to the without MMC and the sham groups, appeared to promote neovascularisation, as evidenced by immunohistochemical analysis of CD31 positive cells responsible for new blood vessel formation (FIG. 6G). The cell sheet groups (both without and with MMC) retained the transplanted cells in the wounds up to 14 days (longest timepoint assessed), as evidenced by immunohistochemical staining of human nuclear antigen (FIG. 6H).

Epidermal thickness analysis revealed no statistically significant differences (p>0.05) between the groups and scar index analysis indicated that the MMC group induced the lowest (p<0.05) scar index.

### Discussion

Scaffold-free tissue engineering aspires to develop 3D tissue surrogates capitalising on the inherent capacity of cells to build tissues and organs. Despite the significant strides that have been achieved over the years and the positive (with respect to both safety and efficacy) preclinical and clinical data, issues associated with very high cell number required that is frequently not available in autologous therapies and the time required to develop a 3D implantable device that not only associated the cell to phenotypic drift, but also the produced device is not really 3D, have jeopardised their wide acceptance and commercialisation. We assessed whether MMC coupled with a temperature-responsive electrospun scaffold can develop safe and functional 3D tissue equivalents using only a fraction of cells and time that traditional scaffold-free approaches utilise.

Starting with the scaffold fabrication element of this work, we demonstrated that the use of the hydrophobic NTBA monomer, at an optimal ratio of 15%, not only decreased the phase transition temperature of the pNIPAM-based electrospun scaffold, but also decreased its response to temperature shift, being stable in wet state, without jeopardising cell attachment and basic cellular functions, as it has been previously demonstrated for pNIPAM-NTBA temperature-responsive films. It is also worth noting that commercially available pNIPAM-based temperature-responsive dishes need relatively long time to detach cell sheets (30-60 min), which rapidly fold and shrink, requiring substantial infrastructure investment for cell sheet manipulation and transplantation and the enhanced ECM deposition (due to MMC), prohibits the release of intact ECM-rich cell layers. Herein, we fabricated for first time pNIPAM-NTBA electrospun scaffolds, and also demonstrated that the produced scaffolds, with a simple temperature shift (from 37° C. to 4° C.), allowed fast (5-10 min), intact and without shrinkage cell sheet detachment, even in the presence of abundant ECM (due to MMC).

Moving into the scaffold-free device development and characterisation, MMC maintained physiological cell function, as judged by basic cell function, growth factor, MMP, SDS-PAGE, immunocytochemistry and histological analyses, as has been shown before for various permanently differentiated and stem cell populations. The significance of this work lays on the fact that using only 50,000 cells/cm² and 10 days of MMC culture time, we developed in one step process a living substitute of more than 300 μm in thickness. To substantiate this, one should consider that traditional temperature-responsive film-derived single cell layer scaffold-free systems require a significantly higher cell number and/or days in culture to produce a significantly thinner device (e.g. subject to cell type, 50,000-612,000 cells/cm² require 4-28 days in culture to produce devices of 10-50 μm in thickness). To increase the thickness of the produced devices, multi-layer cell sheet stacking is used, but this is a multistep process that is notoriously difficult to scale up in reproducible fashion and again requires high cell numbers and prolonged culture times to produce a barely 3D implantable device (e.g. subject to cell type, 3-5 layers of 50,000-1,000,000 cells/cm²/layer require 5-25 days in culture to produce devices of 20-100 m in thickness). It is worth noting that a 350 m in thickness device has been produced after 7-10 days in culture using 9 layers of 200,000 cells/cm²/layer. However, in the absence of sufficient ECM, the very compact conformation of the stacked cell layers results in poor vascularisation, cell death and delamination and polysurgeries (×10) of up to 3 layers (˜80 μm in thickness) are recommended to ultimately result in a neotissue of ˜840 μm in thickness. Of course, such work was conducted in an animal model, as patient distress and healthcare expenditure completely negates such approach in humans. With respect to pNIPAM-based temperature-responsive electrospun-derived scaffold-free systems, again our work is of significant importance, as previous studies have only achieved enzyme-free cell separation and failed to produce intact cell sheets. Only one study has reported intact cell (human mesenchymal stem cells) sheet detachment from a temperature-responsive electrospun hydroxybutyl-chitosan-collagen (the use of collagen avoided shrinkage and increased cell attachment) scaffold after 14 days in culture; however, cell density/cm² and cell sheet thickness were not reported. It is also worth noting, that in the present invention, cells and de novo synthesised and deposited ECM were distributed throughout the thickness of the tissue surrogate. This is again of significant importance, considering that traditional electrospinning setups (like the one used herein) result in very compact electrospun scaffolds with limited cell and tissue infiltration capacity, which gave rise to sacrificial electrospinning. We attribute this 3D in vitro neotissue formation to the enhanced ECM deposition, due to MMC, that infiltrated the electrospun scaffold and encouraged cell migration. After all, in developing tissues, ECM provides paths that support and coordinate cell migration via integrin adhesion complexes that generate traction forces and are responsible for cell migration to interstitial parts of tissues.

With respect to safety and efficacy, using a humanised wound healing model, we demonstrated that the MMC derived scaffold-free devices resulted in accelerated wound closure, collagen-rich neotissue formation and reduced scar area, as judged by histochemical and immunohistochemical analysis. We attribute this significantly improved wound closure to the enhanced, due to MMC, ECM deposition that protected and localised cells and their rich in tropic and reparative factors secretome at the side of implantation. This can be further verified by our in vitro growth factor and MMPs data that demonstrated that MMC created a balance between enhanced growth factor release from the ECM-rich cellular constructs and increased MMP activity, which is in agreement with previous reports showing that the release and activation of matrix-embedded growth factors depends on MMP-mediated proteolysis, which ultimately leads to neo-angiogenesis and tissue regeneration. It is also worth noting that this is the only scaffold-free device that has resulted in such positive therapeutic efficiency with such low number of cells and short culture period. For example, other studies that utilised the same humanised model and hADSCs, showed slower wound closure using 3 layers of 104,000 cells/cm²/layer (for unspecified period of time in culture) or 3 layers of 300,000 cells/cm²/layer cultured for 5 days. Similarly, using the same model, 3 layers of 50,000 human bone marrow stem cells/cm²/layer cultured for 7 days (20 μm in thickness) also resulted in slower wound closure.

### Conclusions

In vitro organogenesis approaches have failed to produce clinically and commercially relevant implantable devices, largely attributed to the prolonged ex vivo culture periods required to develop a barely three-dimensional tissue-like construct that are associated with cell phenotypic drift, loss of cellular function and high manufacturing costs. Herein, macromolecular crowding coupled with a temperature-responsive electrospun scaffold allowed the accelerated development of functional and truly three-dimensional tissue-like surrogates. The proposed approach has the potential to transform cell-assembled regenerative medicine. Scaffold-free in vitro organogenesis exploits the innate ability of cells to synthesise and deposit their own extracellular matrix to fabricate tissue-like assemblies. Unfortunately, traditional cell-assembled tissue engineered concepts require prolonged ex vivo culture periods of very high cell numbers for the development of a borderline three-dimensional implantable device, which are associated with phenotypic drift and high manufacturing costs, thus, hindering their clinical translation and commercialisation. Herein, we report the accelerated (10 days) development of a truly three-dimensional (338.1±42.9 μm) scaffold-free tissue equivalent that promotes fast wound healing and induces neotissue formation composed of mature collagen fibres, using only 50,000 cells/cm² human adipose derived stem cells seeded on an 85:15 poly-N-isopropylacrylamide-N-tert-butylacrylamide temperature-responsive electrospun scaffold and grown under macromolecular crowding conditions (50 μg/ml carrageenan). Our data pave the path for a new era in scaffold-free regenerative medicine.

Tissue Production from Human WS1 Skin Fibroblasts Using Polyacrylic Acid as a Macromolecular Crowder

### Materials & Methods

### Materials

Tissue culture consumables were purchased from Sarstedt (Ireland) and NUNC (Denmark). All chemicals, cell culture media and reagents were purchased from Sigma Aldrich (Ireland), unless otherwise stated. PAA of different molecular weight [450 kDa, 1000 kDa, 4000 kDa] were purchased from Polysciences (USA).

### Solubility Assay

In order to identify working concentrations of the different molecular weight (450, 1000 and 4000 kDa) PAA, different concentrations (100, 500, 1000, 5000, 10,000 and 50,000 μg/ml) of PAA molecular weight were dissolved in standard cell culture media at 37° C.

### Dynamic Light Scattering Analysis

Zeta potential, polydispersity index and hydrodynamic radius were assessed using dynamic light scattering (Zetasizer ZS90, Malvern Instruments, UK). The crowding solutions were prepared in PBS to mimic physiological conditions. Fractional volume occupancy was calculated using the obtained values of hydrodynamic radius for the different MMC agents.

### Cell Culture

Human WS1 skin fibroblasts (ATCC, UK), used between passage 4 and 5, were cultured in Dulbecco's Modified Eagle Medium (Sigma Aldrich, Ireland) supplemented with 10% fetal bovine serum and 1% penicillin streptomycin at 37° C. in a humidified atmosphere of 5% CO2. For MMC experiments, cells were seeded at 25,000 cells/cm2 density and were allowed to attach for 24 h. Subsequently, the media were changed with media containing 100 μM of L-ascorbic acid 2-phosphate sesquimagnesium salt hydrate, to induce collagen synthesis, and PAA. CR at 75 μg/mL and 70/400 Ficoll™ cocktail (37.5/25 mg/ml) were used as positive control. ECM deposition was assessed after 3, 5 and 7 days in culture. The media were changed every alternative day.

### Phase Contrast Microscopy

Phase contrast images were captured using an inverted microscope (Leica Microsystem, Germany) at different time points (3, 5, 7 days) to evaluate the influence of different MMC conditions on cell morphology. Images were processed using ImageJ software (NIH, USA).

### Cell Viability

At the various time points (3, 5 and 7 days), calcein AM (Thermo Fisher Scientific, UK) and ethidium homodimer I (Thermo Fisher Scientific, UK) stainings were performed, as per manufacturer's protocol, to assess the influence of the different crowders on cell viability. Briefly, cells were washed with HBSS and a solution of calcein AM (4 μM) and ethidium homodimer I (2 μM) was added. Cells were incubated at 37° C. and 5% CO2 for 30 minutes after which, fluorescence images were captured with an Olympus IX-81 inverted fluorescence microscope (Olympus Corporation, Japan).

### DNA Quantification

DNA quantification was carried out using Quant-iT™ PicoGreen® dSDNA assay kit (Invitrogen, Ireland) according to the manufacturer's protocol. Briefly, DNA was extracted using three freeze-thaw cycles after adding 250 μl of milliQ water per well (24 well plate). 25 μl of cell suspension was transferred into 96-well plate containing 75 μl of 1×TE buffer. A standard curve was generated using 0, 7.8, 15.6, 31.2, 62.5, 125, 250 and 500 μg/mL DNA concentrations. 100 μl of a 1:200 dilution of Quant-iT™ PicoGreen® reagent was added to each sample and the plate was read using a micro-plate reader (Varioskan Flash, Thermo Scientific, Ireland) with an excitation wavelength of 480 nm and an emission wavelength of 525 nm.

### Cell Metabolic Activity Assessment

An alamarBlue® assay (Invitrogen, USA) was performed to quantify the influence of MMC on metabolic activity of the cells. At the end of culture time points, the cells were washed with Hanks' Balanced Salt solution (HBSS, Sigma Aldrich, Ireland) and then alamarBlue® solution (10% alamarBlue® in HBSS) was added according to the manufacturer's protocol. After 4 h of incubation at 37° C., absorbance was measured at 550 nm and 595 nm using Varioskan Flash spectral scanning multimode reader (Thermo Scientific, UK).

### Sodium Dodecyl Sulphate Polyacrylamide Gel Electrophoresis (SDS PAGE)

Briefly, at the various time points (4, 7 and 10 days), culture media was aspirated, and cell layers were briefly washed with HBSS. Cell layers were then digested with pepsin from porcine gastric mucosa at 0.1 mg/mL in 0.5 M acetic acid (Fischer Scientific, Ireland) at 37° C. for 2 hours under agitation. Cell layers were then scraped and neutralised with 1N sodium hydroxide. Cell layer samples were analysed by SDS-PAGE under non-reducing conditions using a Mini-Protean 3 system (Bio-Rad Laboratories, UK). Bovine collagen type I (100 μg/mL, Symatese Biomateriaux, France) was used as standard for all gels. Staining of the protein bands was performed with SilverQuest™ kit (Thermo Fisher Scientific, UK) following manufacturer's instructions. To quantify the cell-produced collagen type I deposition, the relative densities of collagen α1(I) and α2(I) chains were evaluated with ImageJ and compared to the α1(I) and α2(I) chain bands densities of standard collagen type I. After the quantification, results were normalised to the cell number.

Tissue Production from Human Bone Marrow-Derived Stem Cells Using Polyacrylic Acid as a Macromolecular Crowder

### Materials and Methods

### Cell Culture

Human bone marrow-derived stem cells (RoosterBio Inc, US), used at passage 4, were expanded in Minimum Essential Medium α (MEM α, Thermo Fisher Scientific, UK), supplemented with 1% penicillin streptomycin and 10% foetal bovine serum at 37° C. in a humidified atmosphere of 5% CO2. For MMC experiments, cells were seeded at 25,000 cells/cm² density in 24 or 48 well plates and were allowed to attach for 24 h. Subsequently, the media were changed with media containing 100 μM of L-ascorbic acid 2-phosphate sesquimagnesium salt hydrate and crowders alone or in cocktail (Table 1; indicative suppliers are also provided). ECM deposition was assessed after 5, 8 and 11 days in culture and the media were changed every 3 days.

TABLE 1
Crowders and concentration utilised in this study.

| | | |
|---|---|---|
| Carrageenan (CR) | 50 μg/ml | Sigma, IMCD |
| | | UK Limited |
| Sodium | HA 738 kDa: 1.10 μg/ml | LifecoreBio- |
| Hyaluronate (HA) | HA 1.64 MDa: 1.10 μg/ml | medical |
| Polyvinylpyr- | PVP 360 kDa: 11.34 mg/ml | Sigma |
| rolidone (PVP) | | |
| Ficoll (FC) | FC 400 kDa: 25 mg/ml/FC 70 kDa: | Sigma |
| | 37.5 mg/ml (Cocktail) | |
| Polyacrylic Acid | PAA 1.000.000 kDa: 500.1000 μg/ml | Polyscience |
| (PAA) | PAA 4.000.000 kDa: 500.1000 μg/ml | |

### Phase Contrast Microscopy

Phase contrast images were captured using an inverted microscope (Leica Microsystem, Germany) at different time points (5, 8, 11 days) to evaluate the influence of different MMC conditions on cell morphology. Images were processed using ImageJ software (NIH, USA).

### Cell Viability

At the various time points (5, 8 and 11 days), calcein AM (Thermo Fisher Scientific, UK) and ethidium homodimer I (Thermo Fisher Scientific, UK) stainings were performed, as per manufacturer's protocol, to assess the influence of the different crowders on cell viability. Briefly, cells were washed with HBSS and a solution of calcein AM (4 μM) and ethidium homodimer I (2 μM) was added. Cells were incubated at 37° C. and 5% CO2 for 30 minutes after which, fluorescence images were obtained with an Olympus IX-81 inverted fluorescence microscope (Olympus Corporation, Japan).

### Cell Metabolic Activity

At the different time points (3, 7 and 14 days), the alamarBlue® assay (Thermo Fisher Scientific, UK) was used to evaluate the influence of MMC on cell metabolic activity, as per manufacturer's instructions. Briefly, at each time point, cells were washed with HBSS and a 10% alamarBlue® solution in HBSS was added to the cells. Cells were incubated at 37° C. and 5% CO2 for 3 hours and absorbance was measured at 550 nm and 595 nm with a Varioskan Flash Spectral scanning multimode reader (Thermo Fisher Scientific, UK). Metabolic activity is expressed in terms of percentage of reduced alamarBlue™ normalised to the DNA quantity (μg/ml) obtained from the Quant-iT™ PicoGreen® dsDNA assay.

### Cell Proliferation

Quant-iT™ PicoGreen® dsDNA (Thermo Scientific, UK) assay were performed to quantify the amount of dsDNA in the samples. 250 μL of nucleic acid free water was added per well (48 well plate), the well plate was frozen at −80° C. and three freeze-thaw cycles were performed in order to lyse the cells and extract the DNA. 100 μL of each DNA sample were transferred into a 96-well plate. A standard curve was generated with 0, 100, 200, 375, 500, 1000, 2000 and 4000 μg/mL DNA concentrations. 100 μL of PicoGreen® reagent at 1:200 dilution in 1× Tris-EDTA buffer was added to all standards and samples. Readings were obtained at 480 nm. The DNA concentration was defined as a function of the standard curve and compared at different time points.

### Immunocytochemistry

At each time point (5, 8 and 11 days), cells were briefly washed with HBSS and fixed with 4% paraformaldehyde for 15 minutes at room temperature. Cell were washed again and non-specific site interactions were blocked with 3% bovine serum albumin (BSA) in phosphate buffered saline (PBS) for 30 min. Cells were incubated for 90 minutes at room temperature with the primary antibody (1:500 rabbit anti-collagen type I, R&D; rabbit anti-collagen type III, Abcam, UK), after which, they were washed 3 times with PBS, followed by 30 minutes of incubation at room temperature with the secondary antibody (AlexaFluor® 488 chicken anti rabbit; Thermo Fisher Scientific, UK). Nuclei were counterstained with HOECHST 33342 (Thermo Fisher Scientific, UK) and samples were imaged with Operetta High Content Imaging System.

### Sodium Dodecyl Sulphate-Polyacrylamide Gel Electrophoresis (SDS-PAGE)

At the various time points (5, 8 and 11 days) cell layers were digested with porcine gastric mucosa pepsin (3,200-4,500 units/mg) at 0.1 mg/mL in 0.05 M acetic acid for 2 h at 37° C. with gentle agitation and then neutralised with 0.1 N sodium hydroxide (NaOH). Pepsin-digested samples with 5× sample buffer (0.002% bromophenol blue, 20% glycerol, 2% SDS, 125 mM Tris-HCl, pH 6.8) were loaded onto gels (5% separation gel and 3% stacking gel), after they had been heated at 95° C. for 5 min. The gels were loaded onto a Mini-PROTEAN® electrophoresis system (Tetra Cell, Bio-Rad Laboratories, UK) and ran for approximately 90 minutes (50 V for 30 min and 120 V for 1 h). Bovine type I collagen (0.25 mg/ml, Symatese Biomateriaux, France) was used as a commercial standard control for all gel. SilverQuest™ kit (Invitrogen, UK) was used to stain the protein bands on the SDS-PAGE gel, according to the manufacturer's protocol and the gels were imaged with a HP PrecisionScan Pro scanner (HP, UK). A densitometric analysis has been conducted with the software ImageJ.

### Statistical Analysis

Data are expressed as mean±standard deviation. Statistical analysis was performed using IBM® SPSS® Statistics (IBM, US). One-way analysis of variance (ANOVA) was used for multiple comparisons and Tukey's post hoc test was used for pairwise comparisons after confirming the samples followed a normal distribution (Shapiro-Wilk test) and had equal variances (Bartlett's and Levene's test for homogeneity of variances). When either or both assumptions were violated, non-parametric analysis was conducted using Kruskall-Wallis test for multiple comparisons and Omnibus test for pairwise comparisons. Statistical significance was accepted at p<0.05.

### In-Vivo Wound Closure Study (FIGS. 32 to 39)

### Human Bone Marrow Derived Stem Cell Isolation

Fresh human bone marrow from the iliac crest was purchased from Lonza (UK) and human bone marrow mesenchymal stromal cells (hBM-MSCs) were isolated by seeding the bone marrow on fibronectin coated tissue culture polystyrene flasks with fibroblast culture medium (Lonza, UK) and incubating them in a humidified incubator at 37° C. in the presence of 5% CO₂. The culture medium was replaced with fresh medium every 2 to 3 days and cells were cultured until reached confluency of approximately 80%. hBM-MSCs were harvested from culture flasks using trypsin-ethylenediaminetetraacetic acid, washed with phosphate buffered saline (PBS) and centrifuged at 800 g for 5 min. The cell pellet was suspended in basal medium [α-minimal essential medium (αMEM GlutaMax™, ThermoFisher Scientific, UK) supplemented with 10% foetal bovine serum (FBS) and 1% penicillin/streptomycin (PS)].

### Cell Seeding

Porcine collagen type I scaffolds were provided by Medtronic (France). Scaffolds were cut to size (6 mm in diameter, 0.0129 mm thickness), fixed to the bottoms of 24-well plates, sterilised with 70% ethanol for 2 h and rinsed with sterile PBS. Cells were seeded on collagen scaffolds at passage 3 in basal medium. After 24 h, the media were changed to media with 100 μM of L-ascorbic acid 2-phosphate sesquimagnesium salt hydrate and without/with MMC (carrageenan at 75 μg/ml). Media were changed every 3 days.

### Preclinical Analysis

All animal experiments and procedures were conducted in accordance with Irish laws on animal experimentation and were approved by the Animal Care and Research Ethics Committee of NUI Galway and the Irish Health Products Regulatory Authority (License Number: AE 19125/P051K). A total of 42 female athymic nude mice (7 weeks old) were purchased from Charles River. The animals were housed in individually ventilated cages with controlled temperature and humidity and a 12 h light-dark cycle. Prior to surgery, all animals were housed in groups and postoperatively, animals were housed individually. Animals were allowed access to food and water ad libitum. Prior to surgery and after one week of acclimatisation period, animals were randomly divided into the following six groups: Sham (N=7), Scaffold (N=7), Cells (N=7), 4) Cells+MMC (N=7), Scaffold+Cells (N=7), Scaffold+Cells+MMC (N=7). Cell at passage 3 were cultured for 5 days prior to transplantation. Every animal received perioperative analgesia with a subcutaneous injection of buprenorphine (0.05 mg/kg, Bupaq®, Chanelle Pharma Group, Ireland) 1 h prior to surgical anaesthesia. Anaesthesia was induced and maintained with isoflurane (Iso-Vet®, Chanelle Pharma Group, Ireland). A splinted wound healing model was utilised [59]. Briefly, the surgical field at the back of each mouse was cleaned with iodine scrub and 70% ethanol solution. The skin was folded and two circular full thickness (epidermis, dermis, subcutaneous tissue and panniculus carnosus muscle) wounds of 5 mm diameter were created with a single puncture using a biopsy punch (KAI Medical, Italy). A silicone splint with internal and external diameter of 6 mm and 12 mm, respectively, and 0.5 mm thickness (Grace Bio-Labs, USA) was sutured around every wound to prevent contraction and promote healing by epithelisation. An identical treatment was applied to both wounds of each mouse. Animals received twice per day a subcutaneous injection of buprenorphine (0.05 mg/kg) for post-operative analgesia and once per day a subcutaneous injection of enrofloxacin (5 mg/kg, Baytril®, Bayer, Germany) as antibiotic treatment for 3 days.

### Wound Closure Analysis

Wound closure rate was determined by taking digital pictures of the wounds with a digital camera (Canon, Japan) immediately post-surgery and at days 3, 7, 10 and 14. The planimetric area of the open wounds was measured using ImageJ (NIH, USA). Values were calculated as % of wound closure and calculated as follows: [(area of original wound−area of actual wound)/area of original wound]×100.

### Histological Analysis

After animal euthanasia, skin tissue samples were harvested a fixed in 4% paraformaldehyde for paraffin embedding. 5 m thick cross-sections of skin tissues were prepared at an external vendor (Micro Technical Services, UK). Tissue sections were deparaffinised with 2 immersions in xylene and re-hydrated with descending concentrations of ethanol (100%, 90%, 70%, 0% in distilled water). Sections were stained using haematoxylin-eosin stain and Masson-Goldner's trichrome stain (Carl Roth, Germany), according to the manufacturer's protocols and mounted using DPX mountant. Slides were scanned and images were captured using an Olympus VS120 virtual slide microscope and OlyVIA software and an Olympus BX51 microscope (all Olympus Corporation, Japan).

### Immunohistochemical Analysis

Paraffin sections were dewaxed and re-hydrated as described above. Endogenous peroxidases were blocked by incubating the samples in 3% hydrogen peroxide in 100% methanol for 20 min. Antigen retrieval was carried out in a pressure cooker in 0.01 M Tris-EDTA (pH 9.0). The slides were then incubated for 30 min at room temperature in antigen blocking solution (5% normal goat serum and 0.1% Triton X-100 in PBS). Slides were incubated overnight at 4° C. with the following primary antibodies: rabbit anti-cytokeratin 5, rabbit anti-cytokeratin 14, rabbit anti-CD 31, and mouse anti-human nuclear antigen. Secondary antibodies, biotinylated swine anti-rabbit and biotinylated rabbit anti-mouse (Dako, USA), were added and the slides were further incubated for 1 h at room temperature. For the detection, ABC horseradish peroxidase labelled Vectastain Elite ABC reagent (Vector, UK) was used. Binding sites of primary antibodies were visualised using diaminobenzidine (Dako, UK) as chromogen and all sections were counterstained with haematoxylin. For CD31 staining, slides were incubated with incubated with Alexa Fluor 488 goat anti-mouse secondary antibody and DAPI nuclear stain. Negative controls were prepared for each stain by omitting primary antibodies during incubation, which resulted in no staining. Images were captured using an Olympus VS120 digital scanner and OlyVIA software (both Olympus Corporation, Japan). For CD31 staining, images were acquired using an Olympus IX-81 inverted fluorescence microscope (Olympus Corporation, Japan) and relative fluorescence intensity was analysed with ImageJ (NIH, USA).

### Epidermal Thickness Index

The thickness of the neo-formed epidermis was evaluated with ImageJ (NIH, USA) using Masson-Goldner's trichrome stained histological sections. Beginning from the centre of the wound, 3 non-consecutive sections (100 μm distance from each another) per group, were analysed by randomly selecting 3 high-power fields and performing 5 measurements of the epidermal thickness per field.

### Scar Size Analysis

Scar size analysis was performed as per established protocols [38]. Briefly, scar area was evaluated using Masson-Goldner's trichrome stained histological sections. Beginning from the centre of the wound, 3 non-consecutive sections per group, with a distance of 100 μm, were analysed by randomly selecting 3 high-power fields and performing 5 measurements of the scar size per field.

Scar tissue was outlined using the freeform outline tool in ImageJ (NIH, USA) to produce a pixel-based area measurement, which then converted to μm². Scar area measurements were performed extended to the panniculus carnosus. A positive and predictive relationship was established between dermal thickness and scar area. Scar size was determined by the scar index, which was calculated as follow: scar index (μm)=scar area (μm²)/average dermal thickness (μm). Dermal thickness measurements were obtained using ImageJ (NIH, USA) by drawing a line normal to the average orientation of the epidermal-dermal and dermal-subcutaneous tissue demarcations. 4 dermal thickness measurements were taken per sample, two adjacent to the wound site at 50 μm on either side, and two at a farther distance of 700 μm on either side of the wound.

### Statistical Analysis

Data are expressed as mean±standard deviation. Number of replicates is indicated in each figure legend. Statistical analysis was performed using MINITAB® version 19 (Minitab Inc., USA). One-way analysis of variance (ANOVA) was used for multiple comparisons and Tukey's post hoc test was used for pairwise comparisons after confirming the samples followed a normal distribution (Anderson-Darling test) and had equal variances (Bartlett's and Levene's test for homogeneity of variances). When either or both assumptions were violated, non-parametric analysis was conducted using Kruskal-Wallis test for multiple comparisons and Mann-Whitney test for pairwise comparisons. Statistical significance was accepted at p<0.05.

### REFERENCES

[1] M. Yamato, T. Okano, Cell sheet engineering, Mater Today 7(5) (2004) 42-47.
[2] M. E. Nash, D. Healy, W. M. Carroll, C. Elvira, Y. A. Rochev, Cell and cell sheet recovery from pNIPAm coatings; Motivation and history to present day approaches, J Mater Chem 22(37) (2012) 19376-19389.
[3] N. A. Dzhoyashvili, K. Thompson, A. V. Gorelov, Y. A. Rochev, Film thickness determines cell growth and cell sheet detachment from spin-coated poly(N-isopropylacrylamide) substrates, ACS Appl Mater Interfaces 8(41) (2016) 27564-27572.
[4] D. Healy, M. E. Nash, A. Gorelov, K. Thompson, P. Dockery, S. Beloshapkin, Y. Rochev, Fabrication and application of photocrosslinked, nanometer-scale, physically adsorbed films for tissue culture regeneration, Macromol Biosci 17(2) (2017).
[5] Y. Haraguchi, T. Shimizu, T. Sasagawa, H. Sekine, K. Sakaguchi, T. Kikuchi, W. Sekine, S. Sekiya, M. Yamato, M. Umezu, T. Okano, Fabrication of functional three-dimensional tissues by stacking cell sheets in vitro, Nat Protoc 7(5) (2012) 850-858.
[6] Y. Tsuda, T. Shimizu, M. Yamato, A. Kikuchi, T. Sasagawa, S. Sekiya, J. Kobayashi, G. Chen, T. Okano, Cellular control of tissue architectures using a three-dimensional tissue fabrication technique, Biomaterials 28(33) (2007) 4939-4946[24] J. M. Dang, K. W. Leong, Myogenic induction of aligned mesenchymal stem cell sheets by culture on thermally responsive electrospun nanofibers, Adv Mater 19(19) (2007) 2775-2779.
[7] K. P. Fuller, A. Pandit, D. I. Zeugolis, The multifaceted potential of electro-spinning in regenerative medicine, Pharm Nanotechnol 2(1) (2014) 23-34.
[8] S. Nagarajan, M. Bechelany, N. S. Kalkura, P. Miele, C. P. Bohatier, S. Balme, Chapter 20—Electrospun nanofibers for drug delivery in regenerative medicine, in: S. S. Mohapatra, S. Ranjan, N. Dasgupta, R. K. Mishra, S. Thomas (Eds.), Applications of targeted nano drugs and delivery systems, Elsevier2019, pp. 595-625.
[9] J. M. Dang, K. W. Leong, Myogenic induction of aligned mesenchymal stem cell sheets by culture on thermally responsive electrospun nanofibers, Adv Mater 19(19) (2007) 2775-2779.
[10] A. C. B. Allen, E. Barone, C. O. K. Crosby, L. J. Suggs, J. Zoldan, Electrospun poly(N-isopropyl acrylamide)/poly(caprolactone) fibers for the generation of anisotropic cell sheets, Biomater Sci 5(8) (2017) 1661-1669.
[11] D. M. Wiese, C. C. Ruttan, C. A. Wood, B. N. Ford, L. R. Braid, Accumulating transcriptome drift precedes cell aging in human umbilical cord-derived mesenchymal stromal cells serially cultured to replicative senescence, Stem Cells Transl Med 8(9) (2019) 945-958.
[12] A. Schellenberg, T. Stiehl, P. Horn, S. Joussen, N. Pallua, A. D. Ho, W. Wagner, Population dynamics of mesenchymal stromal cells during culture expansion, Cytotherapy 14(4) (2012) 401-411.
[13] F. Guilak, D. M. Cohen, B. T. Estes, J. M. Gimble, W. Liedtke, C. S. Chen, Control of stem cell fate by physical interactions with the extracellular matrix, Cell stem cell 5(1) (2009) 17-26.
[14] M. Tabaka, T. Kalwarczyk, J. Szymanski, S. Hou, R. Holyst, The effect of macromolecular crowding on mobility of biomolecules, association kinetics, and gene expression in living cells, Front Phys 2(54) (2014).
[15] D. Gaspar, K. P. Fuller, D. I. Zeugolis, Polydispersity and negative charge are key modulators of extracellular matrix deposition under macromolecular crowding conditions, Acta Biomater 88 (2019) 197-210.
[16] Khorasani, H., Zheng, Z., Nguyen, C., Zara, J., Zhang, X., Wang, J., Ting, K., Soo, C., A quantitative approach to scar analysis, Am J Pathol 178(2) (2011) 621-628.

1. A process for the production of a three-dimensional (3D) tissue surrogate (cellular or acellular) comprising of culturing cells in the presence of a three-dimensional scaffold and one or more macromolecular crowders, wherein the macromolecular crowders are large poly-dispersed macromolecules.

2. The process as claimed in claim 1 wherein the scaffold is a sponge, an electrospun scaffold, a hydrogel, a polymer or a temperature-sensitive copolymer fibre scaffold, optionally wherein the scaffold is produced by electrospinning.

3. The process as claimed in claim 1 wherein the temperature sensitive copolymer is selected from Poly-N-isopropylacrylamide-N-tert-butylacrylamide (pNIPAM-NTBA) copolymers, hydroxybutyl chitosan, poly(N-isopropyl-acrylamide) and its copolymers.

4. The process as claimed in claim 1 wherein the scaffold is selected from hydroxyapatite (HA) and tri-calcium phosphate (TCP), polystyrene, poly-l-lactic acid (PLLA), polyglycolic acid (PGA) and poly-dl-lactic-co-glycolic acid (PLGA), collagen, hyaluronic acid, a proteoglycans, alginate-based substrates and chitosan.

5. The process as claimed in claim 1 wherein the macromolecule is a negatively charged or neutral macromolecule.

6. The process as claimed in claim 1 wherein the crowder is selected from the group comprising of: synthetic polymers including polyethylene glycol, polyvinylpyrrolidone, polysodium-4-styrene sulfonate, polyvinyl-alcohol, polyacrylic acid; natural polysaccharides including carrageenan; high, low and non-sulphated dextran; Ficoll, a gum selected from gum Arabica, gum gellan, gum karaya, or gum xanthan, glycosaminoglycans including heparin, heparin sulphate, hyaluronic acid or mixtures thereof.

7. The process as claimed in claim 1 wherein the macromolecular crowder is used in an amount of from about 1 μg/ml culture medium to about 1,000 mg/ml culture medium.

8. The process as claimed in claim 1 in which the macromolecular crowder comprises a natural polysaccharide.

9. The process as claimed in claim 1 in which the macromolecular crowder comprises a carrageenan.

10. The process as claimed in claim 1 in which the scaffold comprises collagen and the cells comprise bone marrow stem cells.

11. The process as claimed in claim 1 in which the scaffold comprises collagen, the cells comprise bone marrow stem cells, and the macromolecular crowder comprises a carrageenan.

12. A three-dimensional (3D) tissue surrogate (cellular or acellular) produced according to a process of claim 1.

13. A method of treating a wound in a mammal, in which a three-dimensional (3D) tissue surrogate produced according to a process of claim 1 is applied to the wound.

14. A method according to claim 13, to improve wound healing or reduce scar formation.

15. A process for the production of a tissue substitute comprising culturing cells in the presence of one or more macromolecular crowders, wherein at least one macromolecular crowder is polyacrylic acid.

16. The process of claim 15, wherein the cells are cultured in the presence of a two dimensional scaffold so that the tissue produced is a 2 dimensional tissue, or a three dimensional scaffold so that the tissue produced is a 3D tissue.

17. The process of claim 15, wherein the one or more macromolecular crowders further comprises one or more of the following: wherein the crowder is selected from the group comprising of: synthetic polymers including polyethylene glycol, polyvinylpyrrolidone, polysodium-4-styrene sulfonate, polyvinyl-alcohol, natural polysaccharides including carrageenan; high, low and non-sulphated dextran; Ficoll, glycosaminoglycans including heparin, heparin sulphate, hyaluronic acid.

18. The process of claim 15 wherein the polyacrylic acid is used in an amount of from about 1 μg/ml culture medium to about 50,000 μg/ml culture medium.

19. The process of claim 15, wherein the polyacrylic acid has an average molecular weight of from about 400 kDa to about 5000 kDa.

20. The process as claimed in claim 1 wherein the cells are selected from permanently differentiated cells such as skin, tendon, cornea, lung, breast fibroblasts; osteoblasts; chondrocytes or stem cells such as bone marrow, adipose-derived, umbilical cord, or engineered cells.

21. The process as claimed in claim 1 wherein the cells are cultured in the presence of culture medium supplemented with a serum, for example fetal bovine serum, human serum, porcine serum, ascorbic acid phosphate, or a combination thereof.

22. A tissue substitute produced by the process of claim 15.
